(19) United States
(12) Patent Application Publication
(10) Pub. No.: US 2003/0191147 A1
(43) Pub. Date: Oct. 9, 2003

(54) Opioid antagonist compositions and dosage forms

(76) Inventors: Barry Sherman, Hillsborough, CA (US); Mary Remien, San Francisco, CA (US); Remi Barbier, San Francisco, CA (US); James W. McGinity, Austin, TX (US)

Correspondence Address: MCANDREWS HELD & MALLOY, LTD, 500 WEST MADISON STREET, SUITE 3400, CHICAGO, IL, 60661

(21) Appl. No.: 10/119,615

(22) Filed: Apr. 9, 2002

(51) Int. Cl. A61K 31/485

(52) U.S. Cl. 514/282

(57) ABSTRACT

The present invention is directed to novel dosage forms, pharmaceutical compositions, kits, and methods of administration of an opioid antagonist in an amount of at least about 0.0001 mg to about or less than about 1.0 mg, including from about 0.0001 mg to less than about 0.5 mg. Solid oral dosage forms are disclosed consisting essentially of an opioid antagonist or alternatively comprising an opioid antagonist and another active ingredient, such as an opioid agonist. Immediate release oral dosage forms are disclosed that release all or a substantial percentage of opioid antagonist, and another active ingredient when present, in a desired time. Concurrent release dosage forms are disclosed that provide concurrent release of an opioid antagonist and another active ingredient.

### CROSS-REFERENCE TO RELATED APPLICATIONS

This application claims the priority of the following U.S. Patent Application Nos. 60/202,227 filed May 5, 2000 (provisional); 60/202,268 filed May 5, 2000 (provisional); 09/756,331 filed Jan. 8, 2001, which is a continuation of 09/566,071 filed May 5, 2000; 60/244,482 filed Oct. 30, 2000 (provisional); 60/245,110 filed Nov. 1, 2000 (provisional); and 60/246,235 filed Nov. 2, 2000 (provisional); and PCT/US00/12493 [WO 00/67739] filed May 5, 2000. The applications cited above are hereby incorporated herein by reference in their entirety to provide continuity of disclosure.

### FIELD OF THE INVENTION

The present invention relates to novel dosage forms and pharmaceutical compositions containing a low dose of an opioid antagonist. The present invention also relates to dosage forms comprising an opioid antagonist and another active pharmaceutical ingredient, such as an opioid agonist.

### BACKGROUND OF THE INVENTION

The pharmacology of opioid antagonist compounds is described in Goodman and Gillman, PHARMACOLOGICAL BASIS OF THERAPEUTICS, Chapter 22, “Opioid Antagonists,” incorporated by reference herein. Opioid antagonists include, but are not limited to, naloxone, cyclazocine, opioid antagonist compounds having the same pentacyclic nucleus as nalmefene, naltrexone, nalorphine, nalbuphine, thebaine, levallorphon, pentazocine, oxymorphine, butorphanol, bupremorphine, levorphanol, meptazinol, dezocine, or pentazocine or their pharmacologically effective salts or esters such as, but not limited to, their hydrochlorides, maleates, tartrates and lactates.

The generally accepted use for opioid antagonists has been to treat overdoses and to prevent abuse of opioid agonists such as heroin or morphine. For these conventional uses, the antagonist such as naloxone or naltrexone is used in relatively high concentrations to effectively block the activity or effects of the opioid agonist. The previously employed high concentration of an opioid antagonist is believed to act antagonistically to the opioid agonist on a biochemical level at opioid receptors on nociceptive neurons.

Naloxone (4,5-epoxy-3,14-dihydroxy-17-(2-prophenyl)morphinan-6-one), the first of the opioid antagonist compounds to be synthesized in 1960, was shortly thereafter discovered to have “pure” antagonist character, i.e., exhibiting virtually no agonist activity. Thus, naloxone became the preferred regime for the treatment of acute narcoticism (the habitual use of narcotics). However, since naloxone exhibited a relatively short duration in the body, it became clear that a longer acting agent having similarly “pure” antagonist character would be even more advantageous. Naltrexone (17-(cyclopropylmethyl)-4,5-epoxy-3,14-dihydroxy-morphinan-6-one) was thus developed in 1965 to fulfill this requirement and was found to have greater potency and longer action than its N-allyl congener, naloxone, as well as activity when given orally. For example, 50 mg dosage forms of naltrexone are marketed as ReVia® in the United States or Trexan in other countries. Nalmefene (6-methylene-6-desoxy-N-cyclopropyl-methyl-14-hydroxydihydroxy-dihydronormorphine) was also developed and is a long acting, orally available, potent opioid antagonist, also with “pure” antagonist properties. These drugs are presently commercially available in certain dosage forms, and are so far as is known, the only pure opioid antagonists which have received governmental approval for administration to humans.

Crain and Shen (BRAIN RESEARCH 757: 176-190 (1997)) have shown that opioid agonists not only activate inhibitory opioid receptors, leading to analgesic activity, but also simultaneously activate a smaller group of excitatory opioid receptors on sensory nerve cells. These effects on the excitatory opioid receptors were proposed to weaken opioid induced analgesia and under certain conditions actually enhance pain. Surprisingly, Crain and Shen (e.g., U.S. Pat. No. 5,512,578, reissued as RE 36,457) showed that co-administration of remarkably low doses of an opioid antagonist, such as naloxone or naltrexone on the order of nanograms/kilogram (ng/kg), when administered to mice with morphine or similar opioid agonists, selectively blocked their effects on excitatory, but not inhibitory, opioid receptors, thus markedly enhancing the analgesic potency of opioid agonists. These discoveries of Crain and Shen have been described in U.S. Pat. Nos. 5,472,943; 5,512,578 reissued as RE 36,457; 5,580,876; and 5,767,125, which are directed to methods for selectively enhancing the analgesic potency of a bimodally-acting opioid agonist and simultaneously attenuating anti-analgesia, hyperalgesia, hyperexcitability, physical dependence and/or tolerance effects associated with the administration of the bimodally-acting opioid agonist.

These methods comprise administering to a subject an analgesic or sub-analgesic amount of a bimodally-acting opioid agonist and an amount of an excitatory opioid receptor antagonist effective to enhance the analgesic potency of the bimodally-acting opioid agonist and attenuate the anti-analgesia, hyperalgesia, hyperexcitability, physical dependence and/or tolerance effects of the bimodally-acting opioid agonist. Also included in these patents are methods for treating pain in a subject by administering to the subject an analgesic or sub-analgesic amount of a bimodally-acting opioid agonist and an amount of an excitatory opioid receptor antagonist effective to enhance the analgesic potency of the bimodally-acting opioid agonist and simultaneously attenuate anti-analgesia, hyperalgesia, hyperexcitability, physical dependence and/or tolerance effects of the bimodally-acting opioid agonist.

Also included in these patents are methods for treating an opiate addict by administering to the opiate addict an amount of an excitatory opioid receptor antagonist either alone or in combination with a bimodally-acting opioid agonist effective to attenuate physical dependence caused by a bimodally-acting opioid agonist and enhance the analgesic potency of a bimodally-acting opioid agonist. Also included are compositions of an analgesic or sub-analgesic amount of a bimodally-acting opioid agonist and an amount of an excitatory opioid receptor antagonist effective to enhance the analgesic potency of the bimodally-acting opioid agonist and attenuate the anti-analgesia, hyperalgesia, hyperexcitability, physical dependence and/or tolerance effects of the bimodally-acting opioid agonist in a subject administered the composition. In all of these studies, the antagonist simultaneously enhanced potency while attenuating such adverse effects.

Two clinical studies on postsurgical patients (Joshi, et. al., ANESTHESIOL. 90: 1007-1011 (1999); Gan et al., ANESTHESIOL. 87: 1075-1081 (1997)) demonstrated that cotreatment of patients with PCA/IV morphine together with a low dose of the opioid antagonist naloxone (IV) or nalmefene (IV) markedly enhanced potency of morphine in varying cumulative daily doses of morphine. Adverse side effects were attenuated in these studies.

A variety of other uses for opioid antagonists have been described or proposed for human treatment where the opioid antagonist is administered in relatively high concentrations, including in amounts similar to that used for treatment of opioid overdose or addiction.

U.S. Pat. No. 6,194,382 B1 describes a method for treating irritable bowel syndrome (IBS) by administration of an amount of an opioid receptor antagonist effective to treat IBS. For example, a daily dose of naltrexone in the range between 0.1 mg/day and about 5 mg/day and of nalmefene in the range between 0.01 mg/day and about 1 mg/day is described.

U.S. Pat. No. 6,187,782 B1 describes morphinan derivatives of a certain formula as compounds having abilities to bind to opioid ε-receptor, which have agonist or antagonist activities. Results of an in vitro antagonist activity assay for eight compounds are shown.

U.S. Pat. No. 6,153,621 describes compositions and methods for treating excitable system disorders, pain and psychiatric disorders with a combination of antagonists of different excitatory systems, such as nicotinic, opioid, serotonergic and andrenergic antagonists, including specifically the combination of the opioid antagonist naltrexone or naloxone and the nicotinic antagonist mecamylamine.

U.S. Pat. No. 6,136,780 describes a method of treating and preventing cancers which are characterized by the presence of zeta receptors, particularly gastrointestinal cancer, by administering an amount of naltrexone, naloxone or [Met⁵]-enkephalin sufficient to block zeta receptors thereby inhibiting or arresting the growth of the cancer. Daily subcutaneous injection of 0.1 mg/kg to mice inoculated with tumor cells resulted in a decrease in tumor incidences and growth.

U.S. Pat. No. 6,110,926 describes an eye drop composition for opioid addiction testing with enhanced stability in the form of a solution of naloxone hydrochloride, (e.g. 0.1-1% w/v) in a mixture of water and at least one pharmaceutically acceptable polyhydric alcohol.

U.S. Pat. No. 6,103,734 describes a multi-step method for suppressing dependence of a patient upon opiates by, among other steps, administration of an opiate antagonist. According to the method, naltrexone may be administered at between 6 mg and 40 mg per hour and naloxone may be administered at between 0.4 mg to 1.5 mg per hour.

U.S. Pat. No. 6,103,258 describes methods for optimizing dopamine, homeostasis during administration of opioid analgesics by administration of an opioid agonist and an amount of a kappa-preferring opioid antagonist, specifically nalmefene, sufficient to inhibit binding of the opioid agonist analgesic at kappa-opioid receptors with only minimal antagonism of the agonist analgesic at mu-opioid receptors. Parenteral administration of nalmefene at 0.00025-0.00015 mg/kg is recommended.

U.S. Pat. No. 6,087,369 describes indole derivative compounds of a certain formula with delta-opioid antagonist activity, and methods with the compounds in immunosuppressive, anti-allergic, anti-inflammatory and brain cell-protecting amounts.

U.S. Pat. No. 6,071,918 describes methods and compositions for treating alcoholism and alcohol dependence by administering a therapeutically effective amount of a synergistic combination of at least one opioid antagonist and at least one selective serotonin reuptake inhibitor.

U.S. Pat. No. 6,034,091 describes a method for treating depression, with claims to the depression associated with alcoholism, by administering a pharmacologically effective dose of opioid antagonist and a pharmacologically effective dose of antidepressant compound.

U.S. Pat. No. 6,026,817 describes a method of treating humans suffering from one or more conditions included within the syndrome of coronary heart disease risk factors (CHDRF) by a stepwise dosing regimen including an opioid antagonist or a drug which substantially equally reduces the specified amounts of catecholamines in higher primary doses and lower maintaining doses.

U.S. Pat. No. 6,004,970 describes a method for treating a person with a nicotine dependency by administering an effective amount of an opioid antagonist and an effective amount of nicotine.

U.S. Pat. No. 6,004,962 describes a multi-step method of rapid opioid detoxification of a patient which includes a step of detoxifying the patient by injecting an opioid antagonist while the patient is sedated, followed by administering antagonist maintenance therapy to the patient.

U.S. Pat. No. 5,972,954 describes a method for preventing or treating opioid induced side effects and non-opioid induced changes in gastrointestinal motility by administering methylnaltrexone, with oral dosages of about 1.0-40.0 mg/kg or as an enterically coated tablet at dosages of about 1.0-80.0 mg/kg.

U.S. Pat. No. 5,958,962 describes a method of treating alcoholism and alcohol dependence with naltrexone and fluoxetine.

U.S. Pat. Nos. 5,922,705 and 5,783,583 describe a method for rapid detoxification of patients addicted to opioid narcotics by infusing nalmefene.

U.S. Pat. No. 5,919,760 describes a method for rapid narcotic detoxification, wherein the acute withdrawal is induced by administering nalmefene, and wherein octreotide is administered in an amount sufficient to alleviate acute and severe diarrhea without precipitating clinically significant bradycardia.

U.S. Pat. No. 5,866,164 describes an osmotic dosage form for lessening the incidence of drug abuse containing in combination an opioid antagonist and an opioid agonist wherein the antagonist and the agonist are maintained as separate in the dosage form. This dosage form provides an osmotically controlled release of the agonist but not the antagonist, characterized by administration of a drug opioid composition free of an antagonist.

U.S. Pat. No. 5,852,032 describes a method of treating a human subject afflicted with nicotine dependence by administering nalmefene to decrease the nicotine dependence.

U.S. Pat. No. 5,817,665 describes a method of and compositions for treating depression with a pharmacologically effective dose of an opioid antagonist having a pentacyclic nucleus structurally analogous to naltrexone, naloxone, nalmefene, nalorphine, nalbuphine, oxymorphone, buprenorphine, thebaine, their pharmacologically effective salts and esters, and combinations thereof, and a pharmacologically effective dose of a compound of one or more nontricyclic antidepressants exhibiting serotonin reuptake activity inhibition in the synapses of the central nervous system, their pharmacologically effective salts and esters, or combinations thereof.

U.S. Pat. No. 5,780,479 describes a therapeutic method of treating an impulse-control disorder, with the exception of trichotillomania, by administering an amount of at least one opioid receptor antagonist effective to reduce or eliminate at least one of the symptoms of the impulse-control disorder.

U.S. Pat. No. 5,714,483 describes antitussive compounds of a certain formula that are delta-opioid antagonists and claims a method for suppressing cough in a subject with an amount of such an antagonist effective to decrease the frequency of coughing. In the case of oral administration, the dose was suggested to be 10 micrograms to 1 gram per day, although test compounds were administered to rats intraperitoneally.

U.S. Pat. No. 5,512,593 describes a method of treating depression with a pharmacologically effective dose of an opioid antagonist selected from the group consisting of naltrexone, naloxone, their pharmacologically effective salts and esters, or combinations thereof, and a pharmacologically effective dose of a compound selected from the group consisting of one or more nontricyclic antidepressants exhibiting serotonin reuptake inhibition in the synapses of the central nervous system, their pharmacologically effective salts and esters, or combinations thereof.

U.S. Pat. No. 5,426,112 describes a method for the treatment and alleviation of pain and addictive behavior in a human with at least one opioid antagonist to effect temporary blockade of the opioid receptor site by administering at least one opioid antagonist in a cumulative amount of less than about 10 mg per day.

U.S. Pat. No. 5,356,900 describes a method of treating humans suffering from chronic herpes virus infections with an essentially pure opiate receptor antagonist having a selectively higher blocking action against Mu opiate receptors than against Delta receptors in an amount which is effective to exert a substantial opiate receptor blocking action against Mu receptors but insufficient to exert such action against Delta receptors.

U.S. Pat. No. 5,352,680 describes delta-opioid receptor antagonists of a certain formula and claims a method for treating the tolerance in a human undergoing administration of an opioid agonist by administering an amount of such an antagonist effective to block or reduce tolerance to an opioid mu reception agonist.

U.S. Pat. No. 5,272,149 describes a method for the treatment of addiction (e.g., to heroin) carried out by reducing the amount of the target addictive agent in the subject, wherein the method comprises multiple specific steps for the successive administration of a plurality of therapeutic agents, each in an amount effective to reduce the physiological level of the target agent in the subject, and wherein the therapeutic agents include naloxone, naltrexone, buprenorphine and hydroxyzine.

U.S. Pat. No. 5,266,574 describes a method of enhancing the wound healing processes by accelerating the growth of wounded tissue and related cells by administering naltrexone in an amount sufficient to continuously blockade the receptor sites of the wounded tissue and related cells.

U.S. Pat. No. 5,025,018 describes a method of inducing kappa-opiate-receptor antagonist activity in a patient suffering from ischemic or traumatic central nervous system injury by administering an effective amount of a kappa-opiate-receptor antagonist suitable to permit the induction of kappa-opiate receptor antagonistic activity. In a preferred embodiment, the kappa opiate receptor antagonist is nalmefene.

U.S. Pat. No. 5,013,739 describes a method of treating humans suffering from chronic fatigue syndrome by the steps of administering by a pharmacologically effective mode to such patient a therapeutically effective dose of an essentially pure opiate receptor antagonist, where the dose corresponding to the therapeutic results produced by Naltrexone in the range from about 1.0 mg to about 10.0 mg.

U.S. Pat. No. 4,935,428 describes a method of treating opiate dependent subjects in which addicts are treated by sublingual administration with a daily dose of 2 to 8 mg buprenorphine for 1 to 4 weeks followed by, as maintenance treatment, the daily simultaneous administration sublingually of 2 to 8 mg buprenorphine and an amount of naltrexone wherein the weights of naltrexone and buprenorphine are within the ratio of 1:4 to 1:1.

U.S. Pat. No. 4,906,637 has the same disclosure as U.S. Pat. No. 5,025,018 and describes a method of treating a patient suffering from ischemic or traumatic brain injury by parenterally administering an effective amount of kappa-opiate receptor antagonist suitable to permit the induction of kappa-opiate-receptor antagonistic activity.

U.S. Pat. No. 4,877,791 describes a method of treating a patient suffering from interstitial cystitis by the daily administration to the patient of from about 1 to about 50 mg of nalmefene or naltrexone.

U.S. Pat. No. 4,863,928 describes a method of treating a patient suffering from an arthritic disease or associated inflammatory disease with daily administration to the patient of from about 1 to about 100 mg of nalmefene or naltrexone.

U.S. Pat. No. 4,857,533 describes a method of treating a patient suffering from an autoimmune disease comprising daily administration of from about 1 to about 100 mg of the narcotic antagonist nalmefene or naltrexone. Oral dosage forms may include generally from about 0.5 to about 50.0 mg of nalmefene or naltrexone per dosage unit.

U.S. Pat. Nos. 4,769,372 and 4,785,000 describe methods of treating patients in chronic pain or suffering from chronic cough without provoking intestinal hypomotility by the oral administration of dosage units comprising in combination opioid analgesics or antitussives and selected opioid antagonists which are substantially devoid of systemic antagonist activity when administered orally.

U.S. Pat. No. 4,774,230 describes a method and compositions for providing glucuronic acid derivatives of opioid antagonists which has a local therapeutic effect in the intestinal tract (e.g. for the treatment of intestinal dysmotility) with a minimum of systemic effects, particularly central nervous system effects. Such derivatives of opioid antagonists are used for intestinal specific drug delivery and in amounts sufficient to provide opioid antagonist to the intestine of the subject, for example, from about 0.1 to 50 mg.

U.S. Pat. No. 4,767,764 describes compounds of a certain formula that are kappa and mu receptor antagonists, but are more selective as kappa-opioid receptor antagonists. Also described are methods for alleviating the effect of an opioid drug and/or exerting an appetite controlling effect by administering such compounds.

U.S. Pat. No. 4,668,685 describes compounds that are substituted benzoate ester prodrug derivatives of 3-hydroxymorphinans of a certain formula, useful in effective analgesic amounts or effective narcotic antagonist amounts.

U.S. Pat. No. 4,600,718 describes a method of treating weight loss disorders consisting essentially of administering to a mammal having a weight loss disorder a daily dosage of an effective amount which consists essentially of at least about 10 milligrams per 37 kilogram body weight of at least one opiate antagonist.

U.S. Pat. No. 4,582,835 describes a method of treating pain by administration of a parenterally or sublingually effective dose of buprenorphine together with an amount of naloxone, for example 0.1 mg per tablet, sufficient to prevent substitution in an opiate dependent subject.

U.S. Pat. No. 4,464,378 describes a method for eliciting an analgesic or narcotic antagonist response in a warm-blooded animal, by nasally administering (a) an analgesically effective amount of morphine, hydromorphone, metopon, oxymorphone, desomorphine, dihydromorphine, levorphanol, cyclazocine, phenazocine, levallorphan, 3-hydroxy-N-methylmorphinan, levophenacylmorphan, metazocine, norlevorphanol, phenomorphan, nalorphine, nalbuphine, buprenorphine, butorphanol or pentazocine, or a nontoxic pharmaceutically acceptable acid addition salt thereof to elicit an analgesic response; or (b) a narcotic antagonist effective amount of naloxone, naltrexone, diprenorphine, nalmexone, cyprenorphine, levallorphan, alazocine, oxilorphan, cyclorphan, nalorphine, nalbuphine, buprenorphine, butorphanol, cyclazocine or pentazocine, or a nontoxic pharmaceutically acceptable acid addition salt thereof to elicit a narcotic antagonist response.

U.S. Pat. No. 4,181,726 describes a method of relieving severe itching by administering an effective dosage of naloxone to a patient suffering from such itching, for example, in dosages of from 0.4 to 1000 mg.

U.S. Pat. No. 3,966,940 describes methods for the treatment of a narcotic-addicted subject by administering an orally effective, but parenterally inactive analgetic composition. Such compositions can contain from about 0.1 mg to about 10 mg of naloxone.

U.S. Pat. No. 3,773,955 describes methods of treating drug-addicted subjects by administering orally effective, but parenterally inactive analgetic compositions comprising the combination of naloxone with opiates such as phenazocine and methadone.

Since opioid antagonists have been manufactured for use conventionally in relatively high concentration dosage forms to treat opioid agonist overdoses and/or to prevent abuse of such agonists (e.g., REVIA® 50 mg naltrexone), compositions and dosage forms of opioid antagonists have not yet been manufactured for use in significantly lower concentrations.

In particular, the commercial manufacture of immediate release dosage forms that provide a dose of a first active agent (e.g., opioid antagonist) and concurrently a substantially lower dose of a second active agent (e.g., opioid agonist) is difficult due to batch-to-batch variability in the amounts of active agent present in individual unit doses. Although a variety of different methods have been developed to address this problem, no particular method is broadly applicable to all combinations of drugs: issues such as undesirable drug/excipient interactions, drug instability, instability of the formulation and unexpected dosage form performance create a continuing demand for formulations and processes tailored to specific drug combinations.

### SUMMARY OF THE INVENTION

The present invention is directed to novel pharmaceutical compositions, dosage forms, and kits with an opioid antagonist. This invention also relates to novel pharmaceutical compositions, dosage forms, and kits with an opioid antagonist and another active pharmaceutical ingredient, for example, an opioid antagonist. The invention further relates to methods for administering to human subjects such pharmaceutical compositions, dosage forms, and kits including an opioid antagonist alone or in combination with another active pharmaceutical ingredient, such as an opioid agonist. Preferred opioid antagonists include naltrexone, nalmefene or naloxone. Particularly preferred is naltrexone. For pharmaceutical compositions, dosage forms, kits and methods according to the invention, an opioid antagonist is provided in an amount from at least about 0.0001 mg to about or less than about 1.0 mg, or at least about 0.001 mg to about or less than about 1.0 mg, or at least about 0.01 mg to about or less than about 1.0 mg, or at least about 0.1 mg to about or less than about 1 mg. Preferred ranges of opioid antagonists also include: from about 0.0001 mg to less than 1.0 mg; from about 0.001 mg to less than 1.0 mg; from about 0.01 mg to less than 1.0 mg; or from about 0.1 mg to less than 1.0 mg. Additional preferred ranges of opioid antagonists include: from about 0.0001 mg to about 0.1 mg; from about 0.001 mg to about 0.1 mg; from about 0.01 mg. to about 0.1 mg; from about 0.001 mg to about 0.1 mg; from about 0.001 mg to about 0.01 mg; or from about 0.01 mg to about 0.1 mg. Further preferred ranges of opioid antagonists include: from at least about 0.0001 to less than about 0.5 mg; from at least about 0.01 to less than about 0.5 mg; or from at least about 0.1 to less than about 0.5 mg.

Each of these dosage forms can be a solid oral dosage form or another dosage form. Each of these pharmaceutical compositions can consist essentially of opioid antagonist and a pharmaceutically acceptable carrier. Each of these kits can consist essentially of a solid oral dosage form of an antagonist and a container. Each of these pharmaceutical compositions or kits can further consist essentially of another active pharmaceutical ingredient, such as an opioid agonist, optionally in a solid oral dosage form. Alternatively, the other active pharmaceutical ingredient, such as an agonist is optionally provided in an injectable, transdermal, transmucosal, oral solution, syrup, elixir or other dosage form.

Each of the pharmaceutical compositions can comprise or consist essentially of opioid antagonist together with another active pharmaceutical ingredient, such as an opioid agonist, formulated with one or more pharmaceutically acceptable materials into a dose form designed for the specific route of administration. Each kit can comprise or consist essentially of one or more containers of medication, each having one or more dosage forms containing either the opioid antagonist alone, combination(s) of the opioid antagonist with the other active pharmaceutical ingredient, such as an opioid agonist, or both. Furthermore the dosage forms and kits may, by their design, constitute a dosing system that provides for the administration of one or both medications in one or more dose forms in a specific regimen necessary to achieve the therapeutic benefits.

The maximum amount of antagonist in the dosage form is 1 mg, alternatively less than 1 mg, alternatively 0.99 mg, alternatively 0.98 mg, alternatively 0.97 mg, alternatively 0.96 mg, alternatively 0.95 mg, alternatively 0.94 mg, alternatively 0.93 mg, alternatively 0.92 mg, alternatively 0.91 mg, alternatively 0.90 mg, alternatively 0.89 mg, alternatively 0.88 mg, alternatively 0.87 mg, alternatively 0.86 mg, alternatively 0.85 mg, alternatively 0.84 mg, alternatively 0.83 mg, alternatively 0.82 mg, alternatively 0.81 mg, alternatively 0.80 mg, alternatively 0.79 mg, alternatively 0.78 mg, alternatively 0.77 mg, alternatively 0.76 mg, alternatively 0.75 mg, alternatively 0.74 mg, alternatively 0.73 mg, alternatively 0.72 mg, alternatively 0.71 mg, alternatively 0.70 mg, alternatively 0.69 mg, alternatively 0.68 mg, alternatively 0.67 mg, alternatively 0.66 mg, alternatively 0.65 mg, alternatively 0.64 mg, alternatively 0.63 mg, alternatively 0.62 mg, alternatively 0.61 mg, alternatively 0.60 mg, alternatively 0.59 mg, alternatively 0.58 mg, alternatively 0.57 mg, alternatively 0.56 mg, alternatively 0,55 mg, alternatively 0.54 mg, alternatively 0.53 mg, alternatively 0.52 mg, alternatively 0.51 mg, alternatively 0.50 mg.

Additionally, the maximum amount of antagonist in the dosage form is less than 0.5 mg, alternatively 0.49 mg, alternatively 0.48 mg, alternatively 0.47 mg, alternatively 0.46 mg, alternatively 0.45 mg, alternatively 0.44 mg, alternatively 0.43 mg, alternatively 0.42 mg, alternatively 0.41 mg, alternatively 0.40 mg, alternatively 0.39 mg, alternatively 0.38 mg, alternatively 0.37 mg, alternatively 0.36 mg, alternatively 0.35 mg, alternatively 0.34 mg, alternatively 0.33 mg, alternatively 0.32 mg, alternatively 0.31 mg, alternatively 0.30 mg, alternatively 0.29 mg, alternatively 0.28 mg, alternatively 0.27 mg, alternatively 0.26 mg, alternatively 0.25 mg, alternatively 0.24 mg, alternatively 0.23 mg, alternatively 0.22 mg, alternatively 0.21 mg, alternatively 0.20 mg, alternatively 0.19 mg, alternatively 0.18 mg, alternatively 0.17 mg, alternatively 0.16 mg, alternatively 0.15 mg, alternatively 0.14 mg, alternatively 0.13 mg, alternatively 0.12 mg, alternatively 0.11 mg, alternatively 0.10 mg, alternatively 0.09 mg, alternatively 0.08 mg, alternatively 0.07 mg, alternatively 0.06 mg, alternatively 0.05 mg, alternatively 0.04 mg, alternatively 0.03 mg, alternatively 0.02 mg, alternatively 0.01 mg, alternatively 0.009 mg, alternatively 0.008 mg, alternatively 0.007 mg, alternatively 0.006 mg, alternatively 0.005 mg, alternatively 0.004 mg, alternatively 0.003 mg, alternatively 0.002 mg, alternatively 0.001 mg, alternatively 0.0009 mg, alternatively 0.0008 mg, alternatively 0.0007 mg, alternatively 0.0006 mg, alternatively 0.0005 mg, alternatively 0.0004 mg, alternatively 0.0003 mg, alternatively 0.0002 mg.

The minimum amount of antagonist in the dosage form is 0.0001 mg, alternatively 0.0002 mg, alternatively 0.0003 mg, alternatively 0.0004 mg, alternatively 0.0005 mg, 0.0006 mg, alternatively 0.0007 mg, alternatively 0.0008 mg, alternatively 0.0009 mg, alternatively 0.001 mg, alternatively 0.002 mg, alternatively 0.003 mg, alternatively 0.004 mg, alternatively 0.005 mg, alternatively 0.006 mg, alternatively 0.007 mg, alternatively 0.008 mg, alternatively 0.009 mg, alternatively 0.01 mg, alternatively 0.011 mg, alternatively 0.012 mg, alternatively 0.013 mg, alternatively 0.014 mg, alternatively 0.015 mg, alternatively 0.016 mg, alternatively 0.017 mg, alternatively 0.018 mg, alternatively 0.019 mg, alternatively 0.02 mg, alternatively 0.021 mg, alternatively 0.022 mg, alternatively 0.023 mg, alternatively 0.024 mg, alternatively 0.025 mg, alternatively 0.026 mg, alternatively 0.027 mg, alternatively 0.028 mg, alternatively 0.029 mg, alternatively 0.03 mg, alternatively 0.031 mg, alternatively 0.032 mg, alternatively 0.033 mg, alternatively 0.034 mg, alternatively 0.035 mg, alternatively 0.036 mg, alternatively 0.037 mg, alternatively 0.038 mg, alternatively 0.039 mg, alternatively 0.04 mg, alternatively 0.041 mg, alternatively 0.042 mg, alternatively 0.043 mg, alternatively 0.044 mg, alternatively 0.045 mg, alternatively 0.046 mg, alternatively 0.047 mg, alternatively 0.048 mg, alternatively 0.049 mg, alternatively 0.05 mg, alternatively 0.051 mg, alternatively 0.052 mg, alternatively 0.053 mg, alternatively 0.054 mg, alternatively 0.055 mg, alternatively 0.056 mg, alternatively 0.057 mg, alternatively 0.058 mg, alternatively 0.059 mg, alternatively 0.06 mg, alternatively 0.061 mg, alternatively 0.062 mg, alternatively 0.063 mg, alternatively 0.064 mg, alternatively 0.065 mg, alternatively 0.066 mg, alternatively 0.067 mg, alternatively 0.068 mg, alternatively 0.069 mg, alternatively 0.07 mg, alternatively 0.071 mg, alternatively 0.072 mg, alternatively 0.073 mg, alternatively 0.074 mg, alternatively 0.075 mg, alternatively 0.076 mg, alternatively 0.077 mg, alternatively 0.078 mg, alternatively 0.079 mg, alternatively 0.08 mg, alternatively 0.081 mg, alternatively 0.082 mg, alternatively 0.083 mg, alternatively 0.084 mg, alternatively 0.085 mg, alternatively 0.086 mg, alternatively 0.087 mg, alternatively 0.088 mg, alternatively 0.089 mg, alternatively 0.09 mg, alternatively 0.091 mg; alternatively 0.092 mg, alternatively 0.093 mg, alternatively 0.094 mg, alternatively 0.095 mg, alternatively 0.096 mg, alternatively 0.097 mg, alternatively 0.098 mg, alternatively 0.099 mg, alternatively 0.1 mg, alternatively 0.11 mg, alternatively 0.12 mg, alternatively 0.13 mg, alternatively 0.14 mg, 0.15 mg, alternatively 0.16 mg, alternatively 0.17 mg, alternatively 0.18 mg, alternatively 0.19 mg, alternatively 0.2 mg, alternatively 0.21 mg, alternatively 0.22 mg, alternatively 0.23 mg, alternatively 0.24 mg, alternatively 0.25 mg, alternatively 0.26 mg, alternatively 0.27 mg, alternatively 0.28 mg, alternatively 0.29 mg, alternatively 0.3 mg, alternatively 0.31 mg, alternatively 0.32 mg, alternatively 0.33 mg, alternatively 0.34 mg, alternatively 0.35 mg, alternatively 0.36 mg, alternatively 0.37 mg, alternatively 0.38 mg, alternatively 0.39 mg alternatively 0.40 mg, alternatively 0.41 mg, alternatively 0.42 mg, alternatively 0.43 mg, alternatively 0.44 mg, alternatively 0.45 mg, alternatively 0.46 mg, alternatively 0.47 mg, alternatively 0.48 mg, alternatively 0.49 mg, alternatively 0.5 mg, alternatively 0.51 mg, alternatively 0.52 mg, alternatively 0.53 mg, alternatively 0.54 mg, alternatively 0.55 mg, alternatively 0.56 mg, alternatively 0.57 mg, alternatively 0.58 mg, alternatively 0.59 mg, alternatively 0.6 mg, alternatively 0.61 mg, alternatively 0.62 mg, alternatively 0.63 mg, alternatively 0.64 mg, alternatively 0.65 mg, alternatively 0.66 mg, alternatively 0.67 mg, alternatively 0.68 mg, alternatively 0.69 mg, alternatively 0.7 mg, alternatively 0.71 mg, alternatively 0.72 mg, alternatively 0.73 mg, alternatively 0.74 mg, alternatively 0.75 mg, alternatively 0.76 mg, alternatively 0.77 mg, alternatively 0.78 mg, alternatively 0.79 mg, alternatively 0.8 mg, alternatively 0.81 mg, alternatively 0.82 mg , alternatively 0.83 mg, alternatively 0.84 mg, alternatively 0.85 mg, alternatively 0.86 mg, alternatively 0.87 mg, alternatively 0.88 mg, alternatively 0.89 mg, alternatively 0.9 mg, alternatively 0.91 mg, alternatively 0.92 mg, alternatively 0.93 mg, alternatively 0.94 mg, alternatively 0.95 mg, alternatively 0.96 mg, alternatively 0.97 mg, alternatively 0.98 mg, alternatively 0.99 mg.

Any minimum amount and any maximum amount of antagonist in the dosage form, as specified above, may be combined to define a range of amounts, providing that the minimum selected is equal to or less than the maximum selected. In one alternative embodiment of the invention the amount of antagonist in the dosage form is less than an effective amount to antagonize an exogenous or endogenous opioid agonist, but such an amount may include an amount that enhances the potency and/or attenuates an adverse effect of the agonist and/or an amount that attenuates tolerance, withdrawal, dependence and/or addiction.

In one alternative embodiment of the invention, the antagonist is present in the form of a pharmaceutically acceptable salt. For example, a dosage form may contain naltrexone hydrochloride as the antagonist. The antagonist can be provided in a form suitable for oral administration. The antagonist can be formulated as a capsule, tablet, pill, or solid sprinkle form.

Yet another aspect of the invention is a method of administering a dose of an opioid antagonist to a human subject by administering a solid dosage form or kit as described above, including, for example, wherein the dose of antagonist is less than an amount effective to antagonize an exogenous or endogenous opioid agonist, (e.g., less than an effective antagonistic amount) but such an amount may include an amount that enhances the potency and/or attenuates an adverse effect of the agonist and/or an amount that attenuates tolerance, withdrawal, dependence, and/or addiction. In one aspect of the invention the method further comprises administering another active pharmaceutical ingredient, such as an opioid agonist, either in a combined dosage form with the antagonist or in a separate dosage form. Such separate agonist dosage form may include solid oral, oral solution, syrup, elixir, injectable, transdermal, transmucosal, or other dosage form.

Even another aspect of the invention is a pharmaceutical kit comprising a dosage form of the opioid antagonist and a container. The kit optionally can also contain a dosage form of an opioid agonist or another active pharmaceutical ingredient. The opioid antagonist and another active pharmaceutical ingredient can be combined in one dosage form or supplied in separate dosage forms that are usable together or sequentially. The opioid antagonist and another active pharmaceutical ingredient can be administered, concurrently, before or after the other.

In another alternative embodiment of the invention, an agonist is present as the other active pharmaceutical ingredient along with the antagonist. The agonist may be present in its original form or in the form of a pharmaceutically acceptable salt. The agonist may be present in an amount that is analgesic or subanalgesic (e.g., non-analgesic) in the human subject. The agonist may also be present in an amount that is anti-analgesic in the human subject. Agonists include alfentanil, buprenorphine, butorphanol, codeine, dezocine, dihydrocodeine, fentanyl, hydrocodone, hydromorphone, leuallorphan, leuorphanol, meperidine, methadone, morphine, nalbuphine, oxycodone, oxmorphone, pentazocine, propoxyphene, and tramadol. The agonist is preferably morphine, hydrocodone, tramadol, or oxycodone, or may include combinations thereof.

In another alternative embodiment of the invention, the opioid antagonist is present with one or more other active pharmaceutical ingredients. For example, other active pharmaceutical ingredients include acetaminophen, steroidal or non-steroidal anti-inflammatory drugs (NSAIDs) such as ibuprofen, COX-1 and/or COX-2 inhibitors such as aspirin, rofecoxib (marketed as VIOXX®), and celecoxib (marketed as CELEBREX™).

Still another aspect of the invention provides an immediate release solid oral dosage form consisting essentially of one or more pharmaceutical excipients, a dose of an opioid agonist and a low dose of an opioid antagonist, wherein the opioid agonist and opioid antagonist are released concurrently when placed in an aqueous environment. Yet another aspect of the invention provides a solid oral dosage form that comprises an opioid agonist and an opioid antagonist and that is essentially free of other active pharmaceutical ingredients.

Yet another aspect of the invention is an immediate release combination solid oral dosage form consisting essentially of an opioid antagonist, another active pharmaceutical ingredient such as an opioid agonist, and one or more pharmaceutical excipients. The opioid antagonist is present in an amount of about 0.0001 to about 1.0 mg, alternatively less than about 1.0 mg, alternatively less than about 0.5 mg. The opioid agonist is present in an amount of about 0.1 to about 300 mg. The opioid antagonist and opioid agonist are released concurrently over a period of less than about 1.5 hours.

Specific embodiments of the invention include those wherein: 1) the dosage form comprises no pharmaceutical excipients that significantly bind, adsorb or complex the opioid antagonist in an aqueous environment; 2) the opioid antagonist is present in an amount ranging from at least about 0.0001 to about 1.0 mg or less than about 1.0 mg or from at least about 0.0001 to less than about 0.5 mg, and an opioid agonist is optimally present in an amount ranging from about 0.1 to about 300 mg; 3) the dosage form comprises at least two pharmaceutical excipients; 4) the dosage form comprises a first pellet of the opioid antagonist coated onto a first nonpareil pellet and optionally a second pellet of the opioid agonist (or another active pharmaceutical ingredient) coated onto a second nonpareil pellet; 5) the dosage form comprises a nonpareil pellet coated with a composition of the opioid antagonist, optionally another active pharmaceutical ingredient such as an opioid agonist, at least one polymer, and at least one plasticizer; 6) the dosage form comprises a first granulation of another the opioid antagonist and a first blend of pharmaceutical excipients and optionally a second granulation of another active pharmaceutical ingredient such as an opioid agonist and a second blend of pharmaceutical excipients; 7) the dosage form comprises a second granulate containing a mixture of a first granulation, the opioid antagonist and at least one pharmaceutical excipient, wherein the first granulation comprises another active pharmaceutical ingredient such as an opioid agonist and at least one pharmaceutical excipient; 8) the dosage form comprises a coated granulation of a mixture of pharmaceutical excipients coated with a binder composition of a binder, the opioid antagonist and optionally another active pharmaceutical ingredient such as an opioid agonist; 9) the dosage form comprises a coated first granulate, wherein the granulate comprises another active pharmaceutical ingredient such as an opioid agonist and a mixture of pharmaceutical excipients and the coating comprises an opioid antagonist; 10) the dosage form comprises spray-dried granules of the opioid antagonist, optionally another active pharmaceutical ingredient such as an opioid agonist and at least one pharmaceutical excipient; 11) the dosage form comprises a soft gelatin capsule filled with a suspension of the opioid antagonist, optionally another active pharmaceutical ingredient such as an opioid agonist and at least one nonaqueous vehicle.

Another aspect of the invention provides methods of making an immediate release solid oral dosage form with a dose of an opioid antagonist and optionally a dose of another active pharmaceutical ingredient such as an opioid agonist, wherein the opioid antagonist or the opioid antagonist and another active pharmaceutical ingredient are greater than 90% released in less than about 45 minutes after exposure to an aqueous environment. Immediate release solid oral dosage forms with antagonist and agonist include those wherein the opioid agonist and opioid antagonist are released concurrently when placed in an aqueous environment.

Other specific embodiments of the invention include those wherein the dosage form is made by: 1) forming a mixture of at least two different coated pellets, wherein the first pellet is made by coating the opioid antagonist onto a first nonpareil pellet and the second pellet is made by coating another active pharmaceutical ingredient onto a second nonpareil pellet; 2) preparing a composition of the opioid antagonist, another active pharmaceutical ingredient, at least one polymer, and at least one plasticizer and applying the composition to a nonpareil pellet; 3) forming a first granulation of an opioid antagonist and a first blend of pharmaceutical excipients, forming a second granulation of another active pharmaceutical ingredient and a second blend of pharmaceutical excipients, and mixing the first and second granulations; 4) forming a first granulation of an active pharmaceutical ingredient and at least one pharmaceutical excipient, and mixing the first granulation, the opioid antagonist and at least one pharmaceutical excipient to form a second granulation; 5) preparing a binder composition of a binder, an opioid antagonist, another active pharmaceutical ingredient and coating a mixture of pharmaceutical excipients with the binder composition to form a coated granulation; 6) coating a composition of an active pharmaceutical ingredient and a mixture of pharmaceutical excipients and with a coating composition of an opioid antagonist; 7) spray-drying a solution of an opioid antagonist, another active pharmaceutical ingredient and at least one pharmaceutical excipient to form spray-dried granules; or 8) filling a soft gelatin capsule with a dispersion of an opioid antagonist, another active pharmaceutical ingredient and at least one non-aqueous vehicle.

### BRIEF DESCRIPTION OF THE DRAWINGS

The following figures form part of the present description and describe exemplary embodiments of the claimed invention. The skilled artisan will, in light of these figures and the description herein, be able to practice the invention without undue experimentation.

FIGS. 1A-1B is a flow diagram illustrating a method of manufacturing naltrexone capsule dosage forms according to a disclosed embodiment.

FIG. 2 depicts an embodiment of an immediate release dosage form with two different types of coated nonpareil pellets.

FIG. 3 depicts an embodiment of an immediate release dosage form with a single type of coated nonpareil pellets.

FIG. 4 depicts an embodiment of the invention of a mixture containing two different types of granules.

FIG. 5 depicts an embodiment of the invention of a coated granule.

FIG. 6 depicts an embodiment of the invention of a granule dispersed within an optional pharmaceutical excipient composition.

FIG. 7 depicts a graph demonstrating the relationship between opioid agonist to opioid antagonist ratio and the amount of a formulation with respect to the total weight of a pharmaceutical composition according to the invention. This relationship holds true for the embodiments of FIGS. 2 and 4.

FIG. 8 depicts a graph demonstrating the relationship between opioid agonist to opioid antagonist ratio and the amount of a first formulation with respect to the total weight of a pharmaceutical composition according to the invention. This relationship holds true for the embodiments of FIGS. 3, 5 and 6.

FIG. 9 depicts an in vitro dissolution profile for the exemplary coated tablets of Example 12.

### DETAILED DESCRIPTION

The present disclosure is directed to novel pharmaceutical compositions of very low doses of opioid antagonists. Novel dosage forms of such antagonists have been manufactured for the first time and administered to human subjects with unexpected benefits. Such novel dosage forms when administered to human subjects enhance the analgesic potency of opioid agonists and/or attenuate (e.g., reduce, block, inhibit or prevent) their adverse side effects and/or attenuate tolerance, withdrawal, dependence and/or addiction. For example, such novel dosage forms simultaneously enhance the analgesic potency of an opioid agonist while attenuating side effects of the agonist, or enhance the analgesic potency of an opioid agonist without attenuating side effects of the agonist, or maintain the analgesic potency of an agonist while attenuating side effects of the agonist, while at the same time or alternatively, attenuate tolerance, withdrawal, dependence and/or addiction. For compositions and methods of the invention that attenuate (e.g., reduce, block or prevent) an adverse effect of the opioid agonist, it is advantageous that the analgesic potency is maintained without increasing or decreasing the cumulative daily dose of agonist.

The present invention includes the manufacture and use of new dosage forms of very small amounts of an opioid antagonist. Clinical trials have yielded surprising effects and benefits in humans. For example, using novel pharmaceutical compositions or dosage forms according to the invention, it was unexpectedly demonstrated that the analgesic potency effects of opioid agonists can be dissociated from their adverse effects in humans. Thus, for the first time, the present invention provides novel pharmaceutical compositions, dosage forms, kits, and methods to dose or treat humans with opioid antagonists. An opioid antagonist is provided in an amount from at least about 0.0001 mg to about or less than about 1.0 mg, or at least about 0.001 mg to about or less than about 1.0 mg, or at least about 0.01 mg to about or less than about 1.0 mg or at least about 0.1 mg to about or less than about 1 mg. Preferred ranges of opioid antagonists also include: from about 0.0001 mg to less than 1.0 mg; from about 0.001 mg to less than 1.0 mg; from about 0.01 mg to less than 1.0 mg; or from about 0.1 mg to less than 1.0 mg. Additional preferred ranges of opioid antagonists include from about 0.0001 mg to about 0.1 mg; from about 0.001 mg to about 0.1 mg; from about 0.01 mg to about 0.1 mg; from about 0.001 mg to about 0.1 mg; from about 0.001 mg to about 0.01 mg; or from about 0.01 mg to about 0.1 mg. Further preferred ranges of opioid antagonists include: from at least about 0.0001 to less than about 0.5 mg; from at least about 0.01 to less than about 0.5 mg; or from at least about 0.1 to less than about 0.5 mg.

The term “opioid” refers to compounds or compositions including metabolites of such compounds or compositions which bind to specific opioid receptors and have agonist (activation) or antagonist (inactivation) effects at these receptors, such as opioid alkaloids, including the agonist morphine and its metabolite morphine-6-glucuronide and the antagonist naltrexone and its metabolite and opioid peptides, including enkephalins, dynorphins and endorphins. The opioid can be present in the present compositions as an opioid base, an opioid pharmaceutically acceptable salt, or a combination thereof. The pharmaceutically acceptable salt embraces an inorganic or an organic salt. Representative salts include hydrobromide, hydrochloride, mucate, succinate, n-oxide, sulfate, malonate, acetate, phosphate dibasic, phosphate monobasic, acetate trihydrate, bi(heplafluorobutyrate); maleate, bi(methylcarbamate), bi(pentafluoropropionate), mesylate, bi(pyridine-3-carboxylate), bi(trifluoroacetate), bitartrate, chlorhydrate, fumarate and sulfate pentahydrate. The term “opiate” refers to drugs derived from opium or related natural or synthetic analogs.

An “opioid receptor agonist” or “opioid agonist” is an opioid compound or composition including any active metabolite of such compound or composition that binds to and activates opioid receptors on nociceptive neurons which mediate pain. Such opioid agonists have analgesic activity (with measurable onset, peak, duration and/or total effect and can produce analgesia. Opioid agonists according to the present invention include: alfentanil, allylprodine, alphaprodine, anileridine, apomorphine, apocodeine, benzylmorphine, bezitramide, buprenorphine, butorphanol, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, nalbuphine, narceine, nicomorphine, norlevorphanol, normethadone, nalorphine, normorphine, norpipanone, ohmefentanyl, opium, oxycodone, oxymorphone, papaveretum, pentazocine, phenadoxone, phenomorphan, phenazocine, phenoperidine, pholcodine, piminodine, piritramide, propheptazine, promedol, profadol, properidine, propiram, propoxyphene, remifentanyl, sufentanyl, tramadol, tilidine, salts thereof, mixtures of any of the foregoing, mixed mu-agonists/antagonists, mu-antagonist combinations, or others known to those skilled in the art. Preferred opioid agonists for human use are morphine, hydrocodone, oxycodone, codeine, fentanyl (and its relatives), hydromorphone, meperidine, methadone, oxymorphone, propoxyphene or tramadol, or mixtures thereof. Particularly preferred contemplated agonists include morphine, hydrocodone, oxycodone or tramadol. Opioid agonists include exogenous or endogenous opioids.

“Bimodally-acting opioid agonists” are opioid agonists that bind to and activate both inhibitory and excitatory opioid receptors on nociceptive neurons which mediate pain. Activation of inhibitory receptors by said agonists causes analgesia. Activation of excitatory receptors by said agonists results in anti-analgesia, hyperexcitability, hyperalgesia, as well as development of physical dependence, tolerance and other undesirable side effects. Bimodally-acting opioid agonists may be identified by measuring the opioid's effect on the action potential duration (APD) of dorsal root ganglion (DRG) neurons in tissue cultures. In this regard, bimodally-acting opioid agonists are compounds which elicit prolongation of the APD of DRG neurons at pM-nM concentrations (i.e. excitatory effects), and shortening of the APD of DRG neurons at μM concentrations (i.e., inhibitory effects).

An “opioid antagonist” is an opioid compound or composition including any active metabolite of such compound or composition that in a sufficient amount attenuates (e.g., blocks, inhibits, prevents or competes with) the action of an opioid agonist. An “effective antagonistic” amount is one which effectively attenuates the analgesic activity of an opioid agonist. For example, 50 mg naltrexone is recognized to be an effective antagonistic amount. Such attenuation is demonstrated when the compound or composition is used in an effective antagonistic dose. An opioid antagonist binds to and blocks (e.g., inhibits) opioid receptors on nociceptive neurons which mediate pain. Opioid antagonists include: naltrexone (marketed in 50 mg dosage forms as ReVia® or Trexan®), naloxone (marketed as Narcan®), nalmefene, methylnaltrexone, naloxone methiodide, nalorphine, naloxonazine, nalide, nalmexone, nalbuphine, nalorphine dinicotinate, naltrindole (NTI), naltrindole isothiocyanate, (NTII), naltriben (NTB), nor-binaltorphimine (nor-BNI), b-funaltrexamine (b-FNA), BNTX, cyprodime, ICI-174,864, LY117413, MR2266, or an opioid antagonist having the same pentacyclic nucleus as nalmefene, naltrexone, nalorphine, nalbuphine, thebaine, levallorphan, oxymorphone, butorphanol, buprenorphine, levorphanol, meptazinol, pentazocine, dezocine, or their pharmacologically effective esters or salts. In some embodiments, the opioid antagonist is naltrexone, nalmefene, naloxone, or mixtures thereof. A specifically contemplated antagonist is naltrexone.

“Excitatory opioid receptor antagonists” are opioids which bind to and act as antagonists to excitatory but not inhibitory opioid receptors on nociceptive neurons which mediate pain. That is, excitatory opioid receptor antagonists are compounds which bind to excitatory opioid receptors and selectively block excitatory opioid receptor functions of nociceptive types of DRG neurons at 1,000 to 10,000-fold lower concentrations than are required to block inhibitory opioid receptor functions in these neurons. Excitatory opioid receptor antagonists may also be identified by measuring their effect on the action potential duration (APD) of dorsal root ganglion (DRG) neurons in tissue cultures. In this regard, excitatory opioid receptor antagonists are compounds that selectively block prolongation of the APD of DRG neurons (i.e., excitatory effects) but not the shortening of the APD of DRG neurons (i.e., inhibitory effects) elicited by a bimodally-acting opioid receptor agonist. Suitable excitatory opioid receptor antagonists include nalmefene, naltrexone, naloxone, etorphine and dihydroetorphine, as well as similarly acting opioid alkaloids and opioid peptides. Preferred excitatory opioid receptor antagonists are naltrexone and nalmefene because of their longer duration of action as compared to naloxone and their greater bioavailability after oral administration.

The opioid antagonists or (if used) opioid agonists or another active pharmaceutical ingredients may be provided in the form of free bases or pharmaceutically acceptable acid addition salts. As used herein, “pharmaceutically acceptable salts” refer to derivatives of the disclosed compounds wherein the therapeutic compound is modified by making acid or base salts thereof. The pharmaceutically acceptable salt embraces an inorganic or an organic salt.

Examples of pharmaceutically acceptable salts include, but are not limited to, mineral or organic acid salts of the opioid antagonist or opioid agonist. The pharmaceutically acceptable salts include the conventional non-toxic salts made, for example, from non-toxic inorganic or organic acids. For example, such conventional non-toxic salts include those derived from inorganic acids such as hydrochloric, hydrobromic, sulfuric, sulfonic, sulfamic, phosphoric, nitric and others known to those skilled in the art; and the salts prepared from organic acids such as amino acids, acetic, propionic, succinic, glycolic, stearic, lactic, malic, malonic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, glucuronic, and other acids. Other pharmaceutically acceptable salts and variants include mucates, phosphate (dibasic), phosphate (monobasic), acetate trihydrate, bi(heptafluorobutyrate), bi(methylcarbamate), bi(pentafluoropropionate), mesylate, bi(pyridine-3-carboxylate), bi(trifluoroacetate), bitartrate, chlorhydrate, and sulfate pentahydrate. An oxide, though not usually referred to by chemists as a salt, is also a “pharmaceutically acceptable salt” for the present purpose. For acidic compounds, the salt may include an amine-based (primary, secondary, tertiary or quaternary amine) counter ion, an alkali metal cation, or a metal cation. Lists of suitable salts are found in texts such as Remington's Pharmaceutical Sciences, 18^{th} Ed. (Alfonso R. Gennaro, ed.; Mack Publishing Company, Easton, Pa., 1990); Remington: the Science and Practice of Pharmacy 19^{th} Ed.( Lippincott, Williams & Wilkins, 1995); Handbook of Pharmaceutical Excipients, 3^{rd} Ed. (Arthur H. Kibbe, ed.; Amer. Pharmaceutical Assoc., 1999); the Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); and Goodman and Gilman's: the Pharmacological Basis of Therapeutics (Louis S. Goodman and Lee E. Limbird, eds.; McGraw Hill, 1992), the disclosures of which are hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The maximum amount of antagonist in the dosage form is 1 mg, alternatively less than 1 mg, alternatively 0.99 mg, alternatively 0.98 mg, alternatively 0.97 mg, alternatively 0.96 mg, alternatively 0.95 mg, alternatively 0.94 mg, alternatively 0.93 mg, alternatively 0.92 mg, alternatively 0.91 mg, alternatively 0.90 mg, alternatively 0.89 mg, alternatively 0.88 mg, alternatively 0.87 mg, alternatively 0.86 mg, alternatively 0.85 mg, alternatively 0.84 mg, alternatively 0.83 mg, alternatively 0.82 mg, alternatively 0.81 mg, alternatively 0.80 mg, alternatively 0.79 mg, alternatively 0.78 mg, alternatively 0.77 mg, alternatively 0.76 mg, alternatively 0.75 mg, alternatively 0.74 mg, alternatively 0.73 mg, alternatively 0.72 mg, alternatively 0.71 mg, alternatively 0.70 mg, alternatively 0.69 mg, alternatively 0.68 mg, alternatively 0.67 mg, alternatively 0.66 mg, alternatively 0.65 mg, alternatively 0.64 mg, alternatively 0.63 mg, alternatively 0.62 mg, alternatively 0.61 mg, alternatively 0.60 mg, alternatively 0.59 mg, alternatively 0.58 mg, alternatively 0.57 mg, alternatively 0.56 mg, alternatively 0.55 mg, alternatively 0.54 mg, alternatively 0.53 mg, alternatively 0.52 mg, alternatively 0.51 mg, alternatively 0.50 mg.

Additionally , the maximum amount of antagonist in the dosage form is less than 0.5 mg, alternatively 0.49 mg, alternatively 0.48 mg, alternatively 0.47 mg, alternatively 0.46 mg, alternatively 0.45 mg, alternatively 0.44 mg, alternatively 0.43 mg, alternatively 0.42 mg, alternatively 0.41 mg, alternatively 0.40 mg, alternatively 0.39 mg, alternatively 0.38 mg, alternatively 0.37 mg, alternatively 0.36 mg, alternatively 0.35 mg, alternatively 0.34 mg, alternatively 0.33 mg, alternatively 0.32 mg, alternatively 0.32 mg, alternatively 0.30 mg, alternatively 0.29 mg, alternatively 0.28 mg, alternatively 0.27 mg, alternatively 0.26 mg, alternatively 0.25 mg, alternatively 0.24 mg, alternatively 0.23 mg, alternatively 0.22 mg, alternatively 0.21 mg, alternatively 0.20 mg, alternatively 0.19 mg, alternatively 0.18 mg, alternatively 0.17 mg, alternatively 0.16 mg, alternatively 0.15 mg, alternatively 0.14 mg, alternatively 0.13 mg, alternatively 0.12 mg, alternatively 0.11 mg, alternatively 0.10 mg, alternatively 0.09 mg, alternatively 0.08 mg, alternatively 0.07 mg, alternatively 0.06 mg, alternatively 0.05 mg, alternatively 0.04 mg, alternatively 0.03 mg, alternatively 0.02 mg, alternatively 0.01 mg, alternatively 0.009 mg, alternatively 0.008 mg, alternatively 0.007 mg, alternatively 0.006 mg, alternatively 0.005 mg, alternatively 0.004 mg, alternatively 0.003 mg, alternatively 0.002 mg, alternatively 0.001 mg, alternatively 0.0009 mg, alternatively 0.0008 mg, alternatively 0.0007 mg, alternatively 0.0006 mg, alternatively 0.0005 mg, alternatively 0.0004 mg, alternatively 0.0003 mg, alternatively 0.0002 mg.

The minimum amount of antagonist in the dosage form is 0.0001 mg, alternatively 0.0002 mg, alternatively 0.0003 mg, alternatively 0.0004 mg, alternatively 0.0005 mg, 0.0006 mg, alternatively 0.0007 mg, alternatively 0.0008 mg, alternatively 0.0009 mg, alternatively 0.001 mg, alternatively 0.002 mg, alternatively 0.003 mg, alternatively 0.004 mg, alternatively 0.005 mg, alternatively 0.006 mg, alternatively 0.007 mg, alternatively 0.008 mg, alternatively 0.009 mg, alternatively 0.01 mg, alternatively 0.011 mg, alternatively 0.012 mg, alternatively 0.013 mg, alternatively 0.014 mg, alternatively 0.015 mg, alternatively 0.016 mg, alternatively 0.017 mg, alternatively 0.018 mg, alternatively 0.019 mg, alternatively 0.02 mg, alternatively 0.021 mg, alternatively 0.022 mg, alternatively 0.023 mg, alternatively 0.024 mg, alternatively 0.025 mg, alternatively 0.026 mg, alternatively 0.027 mg, alternatively 0.028 mg, alternatively 0.029 mg, alternatively 0.03 mg, alternatively 0.031 mg, alternatively 0.032 mg, alternatively 0.033 mg, alternatively 0.034 mg, alternatively 0.035 mg, alternatively 0.036 mg, alternatively 0.037 mg, alternatively 0.038 mg, alternatively 0.039 mg, alternatively 0.04 mg, alternatively 0.041 mg, alternatively 0.042 mg, alternatively 0.043 mg, alternatively 0.044 mg, alternatively 0.045 mg, alternatively 0.046 mg, alternatively 0.047 mg, alternatively 0.048 mg, alternatively 0.049 mg, alternatively 0.05 mg, alternatively 0.051 mg, alternatively 0.052 mg, alternatively 0.053 mg, alternatively 0.054 mg, alternatively 0.055 mg, alternatively 0.056 mg, alternatively 0.057 mg, alternatively 0.058 mg, alternatively 0.059 mg, alternatively 0.06 mg, alternatively 0.061 mg, alternatively 0.062 mg, alternatively 0.063 mg, alternatively 0.064 mg, alternatively 0.065 mg, alternatively 0.066 mg, alternatively 0.067 mg, alternatively 0.068 mg, alternatively 0.069 mg, alternatively 0.07 mg, alternatively 0.071 mg, alternatively 0.072 mg, alternatively 0.073 mg, alternatively 0.074 mg, alternatively 0.075 mg, alternatively 0.076 mg, alternatively 0.077 mg, alternatively 0.078 mg, alternatively 0.079 mg, alternatively 0.08 mg, alternatively 0.081 mg, alternatively 0.082 mg, alternatively 0.083 mg, alternatively 0.084 mg, alternatively 0.085 mg, alternatively 0.086 mg, alternatively 0.087 mg, alternatively 0.088 mg, alternatively 0.089 mg, alternatively 0.09 mg, alternatively 0.091 mg, alternatively 0.092 mg, alternatively 0.093 mg, alternatively 0.094 mg, alternatively 0.095 mg, alternatively 0.096 mg, alternatively 0.097 mg, alternatively 0.098 mg, alternatively 0.099 mg, alternatively 0.1 mg, alternatively 0.11 mg, alternatively 0.12 mg, alternatively 0.13 mg, alternatively 0.14 mg, 0.15 mg, alternatively 0.16 mg, alternatively 0.17 mg, alternatively 0.18 mg, alternatively 0.19 mg, alternatively 0.2 mg, alternatively 0.21 mg, alternatively 0.22 mg, alternatively 0.23 mg, alternatively 0.24 mg, alternatively 0.25 mg, alternatively 0.26 mg, alternatively 0.27 mg, alternatively 0.28 mg, alternatively 0.29 mg, alternatively 0.3 mg, alternatively 0.31 mg, alternatively 0.32 mg, alternatively 0.33 mg, alternatively 0.34 mg, alternatively 0.35 mg, alternatively 0.36 mg, alternatively 0.37 mg, alternatively 0.38 mg, alternatively 0.39 mg alternatively 0.40 mg, alternatively 0.41 mg, alternatively 0.42 mg, alternatively 0.43 mg, alternatively 0.44 mg, alternatively 0.45 mg, alternatively 0.46 mg, alternatively 0.47 mg, alternatively 0.48 mg, alternatively 0.49 mg, alternatively 0.5 mg, alternatively 0.51 mg, alternatively 0.52 alternatively 0.53 mg, alternatively 0.54 mg, alternatively 0.55 mg, alternatively 0.56 mg, alternatively 0.57 mg, alternatively 0.58 mg, alternatively 0.59 mg, alternatively 0.6 mg, alternatively 0.61 mg, alternatively 0.62 mg, alternatively 0.63 mg, alternatively 0.64 mg, alternatively 0.65 mg, alternatively 0.66 mg, alternatively 0.67 mg, alternatively 0.68 mg, alternatively 0.69 mg, alternatively 0.7 mg, alternatively 0.71 mg, alternatively 0.72 mg, alternatively 0.73 mg, alternatively 0.74 mg, alternatively 0.75 mg., alternatively 0.76 mg, alternatively 0.77 mg, alternatively 0.78 mg, alternatively 0.79 mg, alternatively 0.8 mg, alternatively 0.81 mg, alternatively 0.82 mg, alternatively 0.83 mg, alternatively 0.84 mg, alternatively 0.85 mg, alternatively 0.86 mg, alternatively 0.87 mg, alternatively 0.88 mg, alternatively 0.89 mg, alternatively 0.9 mg, alternatively 0.91 mg, alternatively 0.92 mg, alternatively 0.93 mg, alternatively 0.94 mg, alternatively 0.95 mg, alternatively 0.96 mg, alternatively 0.97 mg, alternatively 0.98 mg, alternatively 0.99 mg.

Any minimum and any maximum amount of antagonist in the dosage form, as specified above, may be combined to define a range of amounts, providing that the minimum selected is equal to or less than the maximum selected. In one alternative embodiment of the invention with an opioid antagonist and an opioid agonist, the amount of antagonist in the dosage form is less than an effective amount to antagonize an exogenous or endogenous opioid agonist, but such an amount may include an amount that enhances the potency and/or attenuates an adverse effect of the agonist, and/or attenuates tolerance, withdrawal, dependence and/or addiction.

If used in combination with the opioid antagonist, the opioid agonist is administered in dosage forms containing from about 0.1 to about 300 mg of opioid agonist. The opioid antagonist, alone or in conjunction with opioid agonist, is included in the dosage form in an amount sufficient to produce the desired effect upon the process or condition, including a variety of conditions and diseases in mammals.

When the opioid antagonist is used in combination with the opioid agonist, the amount of the opioid agonist administered may be an analgesic or sub-analgesic amount. As used herein, an “analgesic” amount is amount of the opioid agonist which causes analgesia in a subject administered the opioid agonist alone, and includes standard doses of the agonist which are typically administered to cause analgesia (e.g., mg doses). A “sub-analgesic” amount is an amount which does not cause analgesia in a subject administered the opioid agonist alone, but when used in combination with the opioid antagonist, results in analgesia. A “non-analgesic” amount is an amount which does not cause analgesia when administered to a subject while an “anti-analgesic” amount is an amount which causes algesia (i.e., pain) when administered to a subject. The amount of the opioid antagonist may be an amount effective to enhance the analgesic potency of and/or attenuate the adverse side effects of the opioid agonist, while at the same time or alternatively, attenuate tolerance, withdrawal, dependence and/or addiction. The amount of the opioid antagonist may be less than an effective antagonistic amount or an ineffective antagonistic amount, yet still provide some or all of the foregoing benefits. The optimum amounts of the opioid antagonist administered alone or in combination with an opioid agonist or other therapeutic agent will of course depend upon the particular agonist and antagonist used, the excipient chosen, the route of administration, and/or the pharmacokinetic properties of the patient being treated.

The dosage forms may be made and used in the form of a pharmaceutical preparation, for example, in solid, semisolid, or liquid form, which contains one or more opioid antagonists as an active ingredient, alone or in combination with one or more additional active pharmaceutical ingredients, such as opioid agonists.

Dosage forms according to the invention may comprise a specified active pharmaceutical ingredient, also referred to as an active ingredient or therapeutic agent either alone or in combination with pharmaceutical excipients and other active pharmaceutical ingredient. An “active pharmaceutical ingredient” is defined as any substance or mixture of substances intended to be used in the manufacture of a drug (medicinal) product and that, when used in the production of a drug, becomes an active ingredient of the drug product. Such substances are intended to furnish pharmacological activity or other direct effect in the diagnosis, cure, mitigation, treatment, or prevention of disease or to affect the structure and function of the body. See Draft Consensus Guideline for Good Manufacturing Practice Guide for Active Pharmaceutical Ingredient, International Conference on Harmonisation of Technical Requirements for Registration of Pharmaceuticals for Human Use, release for consultation on Jul. 19, 2000. Other dosage forms may consist essentially of one or more active pharmaceutical ingredients. A dosage form “consisting essentially of” one or more active pharmaceutical ingredients is one that contains only those active pharmaceutical ingredients and one or more pharmaceutical excipients, but does not contain any other active pharmaceutical ingredients. As an example, a dosage form consisting essentially of an opioid antagonist may also contain a binder and a lubricant, but does not contain an active pharmaceutical ingredient other than the opioid antagonist. As another example, a dosage form consisting essentially of an opioid agonist and an opioid antagonist may also contain a binder and a lubricant, but does not contain an active pharmaceutical ingredient other than the opioid agonist and the opioid antagonist.

Any opioid antagonist or opioid agonist may be in admixture with an organic or inorganic carrier or excipient suitable for administration in enteral or parenteral applications, such as orally, topically, transdermally, by inhalation spray, rectally, by subcutaneous, intravenous, intramuscular, subcutaneous, or intrasternal injection or infusion techniques.

The opioid antagonist, alone or in combination with another active pharmaceutical ingredient, such as an opioid agonist, may be compounded, for example, with the usual non-toxic, pharmaceutically acceptable excipients, carriers, diluents or other adjuvants. The choice of adjuvants will depend upon the active ingredients employed, the physical form of the composition, the route of administration, and other factors.

The excipients, binders, carriers, and diluents which can be used include water, glucose, lactose, natural sugars such as sucrose, glucose, or corn sweeteners, sorbitol, natural and synthetic gums such as gum acacia, tragacanth, sodium alginate, and gum arabic, gelatin, mannitol, starches such as starch paste, corn starch, or potato starch, magnesium trisilicate, talc, keratin, colloidal silica, urea, stearic acid, magnesium stearate, dibasic calcium phosphate, crystalline cellulose, methyl cellulose, carboxymethyl cellulose, polyethylene glycol, waxes, glycerin, and saline solution, among others.

Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

The dosage forms can also comprise one or more acidifying agents, adsorbents, alkalizing agents, antiadherents, antioxidants, binders, buffering agents, colorants, complexing agents, diluents or fillers, direct compression excipients, disintegrants, flavorants, fragrances, glidants, lubricants, opaquants, plasticizers, polishing agents, preservatives, sweetening agents, or other ingredients known for use in pharmaceutical preparations.

As used herein, the term “acidifying agent” is intended to mean a compound used to provide an acidic medium for product stability. Such compounds include, by way of example and without limitation, acetic acid, amino acid, citric acid, fumaric acid and other alpha hydroxy acids, hydrochloric acid, ascorbic acid, nitric acid, phosphoric acid, and others known to those skilled in the art.

As used herein, the term “adsorbent” is intended to mean an agent capable of holding other molecules onto its surface by physical or chemical (chemisorption) means. Such compounds include, by way of example and without limitation, powdered and activated charcoal, zeolites, and other materials known to one of ordinary skill in the art.

As used herein, the term “alkalizing agent” is intended to mean a compound used to provide an alkaline medium for product stability. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those skilled in the art.

As used herein, the term “antiadherent” is intended to mean an agent that prevents the sticking of solid dosage formulation ingredients to punches and dies in a tableting machine during production. Such compounds include, by way of example and without limitation, magnesium stearate, talc, calcium stearate, glyceryl behenate, PEG, hydrogenated vegetable oil, mineral oil, stearic acid and other materials known to one of ordinary skill in the art.

As used herein, the term “antioxidant” is intended to mean an agent which inhibits oxidation and thus is used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art.

As used herein, the term “binder” is intended to mean a substance used to cause adhesion of powder particles in solid dosage formulations. Such compounds include, by way of example and without limitation, acacia, alginic acid, carboxymethylcellulose sodium, poly(vinylpyrrolidone), compressible sugar (e.g., NuTab), ethylcellulose, gelatin, liquid glucose, methylcellulose, povidone and pregelatinized starch and other materials known to one of ordinary skill in the art.

When needed, binders may also be included in the dosage forms. Exemplary binders include acacia, tragacanth, gelatin, starch, cellulose materials such as methyl cellulose, HPMC, HPC, HEC and sodium carboxy methyl cellulose, alginic acids and salts thereof, polyethylene glycol, guar gum, polysaccharide, bentonites, sugars, invert sugars, poloxamers (PLURONIC™ F68, PLURONIC™ F127), collagen, albumin, gelatin, cellulosics in nonaqueous solvents, combinations thereof and others known to those skilled in the art. Other binders include, for example, polypropylene glycol, polyoxyethylene-polypropylene copolymer, polyethylene ester, polyethylene sorbitan ester, polyethylene oxide, combinations thereof and other materials known to one of ordinary skill in the art.

As used herein, the term “buffering agent” is intended to mean a compound used to resist changes in pH upon dilution or addition of acid or alkali. Such compounds include, by way of example and without limitation, potassium metaphosphate, potassium phosphate, monobasic sodium acetate and sodium citrate anhydrous and dihydrate and other materials known to one of ordinary skill in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol, sucrose, and other materials known to one of ordinary skill in the art.

As used herein, the term “diluent” or “filler” is intended to mean an inert substance used to create the desired bulk, flow properties, and compression characteristics in the preparation of solid dosage forms. Such compounds include, by way of example and without limitation, dibasic calcium phosphate, kaolin, lactose, dextrose, magnesium carbonate, sucrose, mannitol, microcrystalline cellulose, powdered cellulose, precipitated calcium carbonate, calcium sulfate, sorbitol, and starch and other materials known to one of ordinary skill in the art.

As used herein, the term “direct compression excipient” is intended to mean a compound used in compressed solid dosage forms. Such compounds include, by way of example and without limitation, dibasic calcium phosphate (e.g., Ditab) and other materials known to one of ordinary skill in the art.

As used herein, the term “disintegrant” is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starches thereof, sweeteners, clays such as bentonite, microcrystalline cellulose (e.g., Avicel), methyl cellulose, carboxymethylcellulose calcium, sodium carboxymethylcellulose, alginic acid, sodium alginate, cellulose polyacrilin potassium (e.g., Amberlite), alginates, sodium starch glycolate, gums, agar, guar, locust bean, karaya, xanthan, pectin, tragacanth, agar, bentonite, and other materials known to one of ordinary skill in the art.

As used herein, the term “glidant” is intended to mean an agent used in solid dosage formulations to promote flowability of the solid mass. Such compounds include, by way of example and without limitation, colloidal silica, cornstarch, talc, calcium silicate, magnesium silicate, colloidal silicon, tribasic calcium phosphate, silicon hydrogel and other materials known to one of ordinary skill in the art.

As used herein, the term “lubricant” is intended to mean a substance used in solid dosage formulations to reduce friction during compression. Such compounds include, by way of example and without limitation, sodium oleate, sodium stearate, calcium stearate, zinc stearate, magnesium stearate, polyethylene glycol, talc, mineral oil, stearic acid, sodium benzoate, sodium acetate, sodium chloride, and other materials known to one of ordinary skill in the art.

As used herein, the term “opaquant” is intended to mean a compound used to render a coating opaque. An opaquant may be used alone or in combination with a colorant. Such compounds include, by way of example and without limitation, titanium dioxide, talc and other materials known to one of ordinary skill in the art.

As used herein, the term “polishing agent” is intended to mean a compound used to impart an attractive sheen to solid dosage forms. Such compounds include, by way of example and without limitation, carnauba wax, white wax and other materials known to one of ordinary skill in the art.

As used herein, the term “colorant” is intended to mean a compound used to impart color to solid (e.g., tablets) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, ferric oxide, other FD&C dyes and natural coloring agents such as grape skin extract, beet red powder, beta-carotene, annato, carmine, turmeric, paprika, and other materials known to one of ordinary skill in the art. The amount of coloring agent used will vary as desired.

As used herein, the term “flavorant” is intended to mean a compound used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors which have been found to be particularly useful include commercially available orange, grape, cherry and bubble gum flavors and mixtures thereof. The amount of flavoring may depend on a number of factors, including the organoleptic effect desired. Flavors will be present in any amount as desired by those skilled in the art. Particularly contemplated flavors are the grape and cherry flavors and citrus flavors such as orange.

Complexing agents include for example EDTA disodium or its other salts and other agents known to one of ordinary skill in the art.

Exemplary fragrances include those generally accepted as FD&C grade.

Exemplary preservatives include materials that inhibit bacterial growth, such as Nipagin, Nipasol, alcohol, antimicrobial agents, benzoic acid, sodium benzoate, benzyl alcohol, sorbic acid, parabens, isopropyl alcohol and others known to one of ordinary skill in the art.

The solid dosage forms of the invention can also employ one or more surface active agents or cosolvents that improve wetting or disintegration of the core and/or layer of the solid dosage form.

Plasticizers can also be included in the tablets to modify the properties and characteristics of the polymers used in the coats or core of the tablets. As used herein, the term “plasticizer” includes all compounds capable of plasticizing or softening a polymer or binder used in invention. The plasticizer should be able to lower the melting temperature or glass transition temperature (softening point temperature) of the polymer or binder. Plasticizers, such as low molecular weight PEG, generally broaden the average molecular weight of a polymer in which they are included thereby lowering its glass transition temperature or softening point. Plasticizers also generally reduce the viscosity of a polymer. It is possible the plasticizer will impart some particularly advantageous physical properties to the dosage form of the invention.

Plasticizers useful in the invention can include, by way of example and without limitation, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, acetyltributylcitrate, triethyl citrate, acetyl triethyl citrate, tributyl citrate and allyl glycolate. All such plasticizers are commercially available from sources such as Aldrich or Sigma Chemical Co. It is also contemplated and within the scope of the invention, that a combination of plasticizers may be used in the present formulation. The PEG based plasticizers are available commercially or can be made by a variety of methods, such as disclosed in Poly(ethylene glycol) Chemistry: Biotechnical and Biomedical Applications (J. M. Harris, Ed.; Plenum Press, NY) the disclosure of which is hereby incorporated by reference.

The solid dosage forms of the invention can also include oils, for example, fixed oils, such as peanut oil, sesame oil, cottonseed oil, corn oil and olive oil; fatty acids, such as oleic acid, stearic acid and isostearic acid; and fatty acid esters, such as ethyl oleate, isopropyl myristate, fatty acid glycerides and acetylated fatty acid glycerides. It can also be mixed with alcohols, such as ethanol, isopropanol, hexadecyl alcohol, glycerol and propylene glycol; with glycerol ketals, such as 2,2-dimethyl-1,3-dioxolane-4-methanol; with ethers, such as poly(ethyleneglycol) 450, with petroleum hydrocarbons, such as mineral oil and petrolatum; with water, or with mixtures thereof; with or without the addition of a pharmaceutically suitable surfactant, suspending agent or emulsifying agent.

Soaps and synthetic detergents may be employed as surfactants and as vehicles for the solid pharmaceutical compositions. Suitable soaps include fatty acid alkali metal, ammonium, and triethanolamine salts. Suitable detergents include cationic detergents, for example, dimethyl dialkyl ammonium halides, alkyl pyridinium halides, and alkylamine acetates; anionic detergents, for example, alkyl, aryl and olefin sulfonates, alkyl, olefin, ether and monoglyceride sulfates, and sulfosuccinates; nonionic detergents, for example, fatty amine oxides, fatty acid alkanolamides, and poly(oxyethylene)-block-poly(oxypropylene) copolymers; and amphoteric detergents, for example, alkyl β-aminopropionates and 2-alkylimidazoline quaternary ammonium salts; and others known to one of ordinary skill in the art; and mixtures thereof.

A water soluble coat or layer can be formed to surround a solid dosage form or a portion thereof. The water soluble coat or layer can either be inert or drug-containing. Such a coat or layer will generally comprise an inert and non-toxic material which is at least partially, and optionally substantially completely, soluble or erodible in an environment of use. Selection of suitable materials will depend upon the desired behavior of the dosage form. A rapidly dissolving coat or layer will be soluble in the buccal cavity and/or upper GI tract, such as the stomach, duodenum, jejunum or upper small intestines. Exemplary materials are disclosed in U.S. Pat. No. 4,576,604 to Guittard et al. and U.S. Pat. No. 4,673,405 to Guittard et al., and U.S. Pat. No. 6,004,582 to Faour et al. and the text Pharmaceutical Dosage Forms: Tablets Volume I, 2^{nd} Edition. (A. Lieberman. ed. 1989, Marcel Dekker, Inc.), the disclosures of which are hereby incorporated by reference. In some embodiments, the rapidly dissolving coat or layer will be soluble in saliva, gastric juices, or acidic fluids.

Materials which are suitable for making the water soluble coat or layer include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; croscarmellose sodium; other cellulose polymers such as sodium carboxymethylcellulose; and other materials known to those skilled in the art. Other lamina-forming materials that can be used for this purpose include poly(vinyl alcohol), poly(ethylene oxide), gelatin, glucose and saccharides. The water soluble coating can comprise other pharmaceutical excipients that may or may not alter the way in which the water soluble coating behaves. The artisan of ordinary skill will recognize that the above-noted materials include film-forming polymers.

A water soluble coat or layer can also comprise hydroxypropyl methylcellulose, which is supplied by Dow under its Methocel E-15 trademark. The materials can be prepared in solutions having different concentrations of polymer according to the desired solution viscosity. For example, a 2% W/V aqueous solution of Methocel™ E-15 has a viscosity of about 13-18 cps at 20° C.

For transdermal administration, the compounds may be combined with skin penetration enhancers such as propylene glycol, polyethylene glycol, isopropanol, ethanol, oleic acid, N-methylpyrrolidone, or others known to those skilled in the art, which increase the permeability of the skin to the compounds, and permit the compounds to penetrate through the skin and into the bloodstream. The compound/enhancer compositions also may be combined additionally with a polymeric substance such as ethylcellulose, hydroxypropyl cellulose, ethylene/vinylacetate, or others known to those skilled in the art, to provide the composition in gel form, which can be dissolved in solvent such as methylene chloride, evaporated to the desired viscosity, and then applied to backing material to provide a patch.

For intravenous, intramuscular, or subcutaneous administration, the active ingredients may be combined with a sterile aqueous solution which is preferably isotonic with the blood of the recipient. Such formulations may be prepared by dissolving one or more solid active ingredients in water containing physiologically compatible substances such as sodium chloride, glycine, or others known to those skilled in the art, and/or having a buffered pH compatible with physiological conditions to produce an aqueous solution, and/or rendering the solution sterile. The formulations may be present in unit dose containers such as sealed ampoules or vials.

A solid dosage form of the invention can be coated with a finish coat as is commonly done in the art to provide the desired shine, color, taste or other aesthetic characteristics. Materials suitable for preparing the finish coat are well known in the art and found in the disclosures of many of the references cited and incorporated by reference herein.

Various other components, in some cases not otherwise listed above, can be added to the present formulation for optimization of a desired active agent release profile including, by way of example and without limitation, glycerylmonostearate, nylon, cellulose acetate butyrate, d,1-poly(lactic acid), 1,6-hexanediamine, diethylenetriamine, starches, derivatized starches, acetylated monoglycerides, gelatin coacervates, poly (styrene-maleic acid) copolymer, glycowax, castor wax, stearyl alcohol, glycerol palmitostearate, poly(ethylene), poly(vinyl acetate), poly(vinyl chloride), 1,3-butylene-glycoldimethacrylate, ethyleneglycol-dimethacrylate and methacrylate hydrogels.

It should be understood that compounds used in the art of pharmaceutical formulation generally serve a variety of functions or purposes. Thus, whether a compound named herein is mentioned only once or is used to define more than one term herein, its purpose or function should not be construed as being limited solely to the named purpose(s) or function(s).

For preparing solid compositions such as tablets, the opioid antagonist, alone or in conjunction with an opioid agonist, is mixed with a pharmaceutical carrier or excipient, such as conventional tableting ingredients and other pharmaceutical diluents, such as water, to form a solid preformulation composition containing a homogeneous mixture of a compound or a non-toxic pharmaceutically acceptable salt thereof. When referring to these preformulation compositions as homogeneous, it is meant that the opioid antagonist, alone or in conjunction with an opioid agonist, is dispersed evenly throughout the composition so that the composition may be readily subdivided into equally effective unit dosage forms such as capsules, tablets, caplets, or pills. This solid preformulation composition is then subdivided into unit dosage forms of the type described above containing the above-stated dose of an opioid antagonist, alone or in combination with opioid agonist.

Solid compositions of the opioid antagonist alone may be administered in combination with any other therapeutic agent(s), including, but not limited to, opioid agonists.

Capsules, tablets, caplets, or pills of the novel pharmaceutical composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Controlled release (e.g., slow-release or sustained-release) dosage forms, as well as immediate release dosage forms are specifically contemplated. Controlled release compositions in liquid forms in which a therapeutic agent may be incorporated for administration orally or by injection are also contemplated.

The pharmaceutical compositions or dosage forms of this invention may be used in the form of a pharmaceutical preparation which contains one or more opioid antagonists alone or in combination with one or more opioid agonists or other active pharmaceutical ingredient.

It has been unexpectedly discovered that some opioid antagonists undesirably bind significantly to certain pharmaceutical excipients in an environment of use. Those pharmaceutical excipients generally cause an incomplete amount of the opioid antagonist to be released from an immediate release dosage form, within a particular time allotted for release in a dissolution test or in clinical use. For example, when naltrexone hydrochloride in solution was mixed with croscarmellose sodium in suspension, the croscarmellose sodium bound more than 90% of the naltrexone hydrochloride. Morphine sulfate also bound to this excipient. However, when lactose, dibasic calcium phosphate, dextrose, or sucrose were tested in combination with the above drugs, the drugs were not bound significantly by the excipients. Accordingly, some embodiments of the invention include specific combinations of opioid antagonist and pharmaceutical excipient, wherein the excipient does not bind the opioid antagonist to a significant degree in an environment of use. Other embodiments include combinations of opioid agonist, opioid antagonists, and pharmaceutical excipients, wherein the excipients binds the opioid agonist and opioid antagonist to effectively the same degree, so that they are released concurrently, sequentially and/or have substantially the same dissolution rates.

The combination dosage forms of the present invention can be formulated to provide a concurrent release of the opioid agonist and opioid antagonist generally throughout at least a majority of the delivery profile for the formulation. As used herein, the terms “concurrent release” and “released concurrently” mean that the agonist and antagonist are released in in vitro dissolution assays in an overlapping manner. The respective beginnings of release of each agent can but need not necessarily be simultaneous. Concurrent release will occur when the majority of the release of the first agent overlap a majority of release of the second agent. According to one exemplary embodiment, release of the agonist and antagonist begins and ends at approximately the same time. In some embodiments of formulations comprising an opioid antagonist and an opioid agonist, the dissolution rates of the antagonist and the agonist are substantially the same. A desired portion of each active pharmaceutical ingredient may be released within a desired time. The desired portions may be, for example, 5%, 50% or 90%, or some other percentage. The desired time may be, for example, 10 minutes, 20 minutes, 30 minutes or 45 minutes.

Generally, the entire charge of each active pharmaceutical ingredient is released in less than 120 min, less than 90 min, less than 60 min, less than 45 min, less than 30 min, less than 20 min or less than 10 min. Preferably, the entire charge of each active pharmaceutical ingredient is released in less than 45 minutes.

According to a specific embodiment of the invention, each active pharmaceutical ingredient is released as follows:

| Time (in minutes) | Amount Released (% wt.) |
|---|---|
| 0 | 0 |
| 5 | ≧35 and ≦75 |
| 10 | ≧50 and ≦90 |
| 15 | ≧75 and ≦95 |
| 30 | 90 |

Coated tablets, beads, pellets or granules can be made to provide an immediate and/or a concurrent release of an opioid antagonist and an opioid agonist. Such dosage forms are made according to the compositions and dosage forms described herein, for example as described in the examples. A coated solid substrate will independently include the opioid antagonist and/or the opioid agonist in the solid substrate or the coat. For example, specific embodiments include those wherein: 1) another active pharmaceutical ingredient, such as an opioid agonist, is in the core and the opioid antagonist is in the coat that at least partially surrounds the core; 2) another active pharmaceutical ingredient, such as an opioid agonist, and an opioid antagonist are both in the core; 3) another active pharmaceutical ingredient, such as an opioid agonist, and opioid antagonist are both in a coat that at least partially surrounds an inert core; and 4) the opioid antagonist is in the core and another active pharmaceutical ingredient, such as an opioid agonist, is in the coat that at least partially surrounds the core.

The term “unit dose” is used herein to mean a dosage form containing a quantity of the therapeutic compounds, said quantity being such that one or more predetermined units may be provided as a single therapeutic administration. Depending upon the specific combination and amounts of agonist and antagonist included within the dosage form, an improved, additive or synergistic therapeutic effect will be observed. An improved therapeutic effect is one wherein the antagonist enhances the therapeutic effect, such as analgesic effect, provided by the agonist alone. An additive therapeutic effect is one wherein each of the antagonist and the agonist possesses a common therapeutic effect, and the combination of the two drugs provides an overall therapeutic effect that approximates the sum of their individual therapeutic effects. A synergistic therapeutic effect is one wherein the combination of the two drugs provides an overall therapeutic effect that is greater than the sum of their individual therapeutic effects.

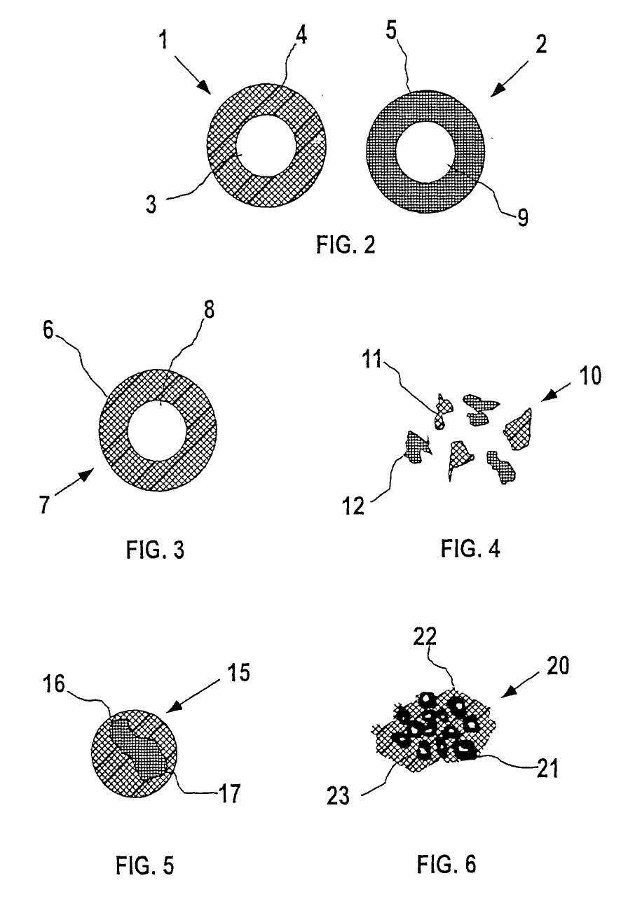

FIG. 2 depicts a non-limiting embodiment of the invention: a pharmaceutical composition of coated nonpareil solid substrates 1 and 2, such as pellets or beads. The coated pellet 1 comprises an inert nonpareil core 3 of one or more pharmaceutical excipients and a coat 4 of a low dose of an opioid antagonist and one or more pharmaceutical excipients. The coated pellet 2 comprises an inert nonpareil core 9 of one or more pharmaceutical excipients and a coat 5 of a low dose of an active pharmaceutical ingredient (in this case, an opioid agonist) and one or more pharmaceutical excipients. The pharmaceutical excipients can be selected independently at each occurrence. Likewise, the cores 3 and 9 can comprise the same or different ingredients. The two different types of coated nonpareils can be filled into a capsule, such as a hard gelatin capsule, or compressed into a tablet core. The coated nonpareils are optionally mixed with one or more other pharmaceutical excipients prior to being filled into the capsule or compressed into the tablet core.

The coated nonpareils 1 and 2 are made according to the process of Example 10 and generally by forming a mixture of at least two different coated pellets, wherein the first pellet is made by coating the opioid antagonist onto a first nonpareil pellet and the second pellet is made by coating the opioid agonist onto a second nonpareil pellet.

When included in a capsule or tablet, the overall amount of each drug present in the respective dosage form will depend upon the amount of drug needed or useful to provide the desired therapeutic response.

FIG. 3 depicts a pharmaceutical composition of a coated nonpareil bead 7 of an inert nonpareil core 8 of one or more pharmaceutical excipients and a coat 6 of a low dose of an opioid antagonist, a dose of another pharmaceutical ingredient (in this case, an opioid agonist) and one or more pharmaceutical excipients. The pharmaceutical excipients(s) can be selected independently at each occurrence. The coated nonpareil can be filled into a capsule, such as a hard gelatin capsule, or compressed into a tablet core. The coated nonpareil is optionally mixed with one or more other pharmaceutical excipients prior to being filled into the capsule or compressed into the tablet core.

The coated nonpareil bead 7 is made according to the process of Example 11 and generally by preparing a composition of the opioid antagonist, the opioid agonist, at least one polymer or film-forming material, and optionally a plasticizer and applying the composition to a nonpareil pellet.

FIG. 4 depicts a mixed granulation 10 of a first granulation 11 of an opioid antagonist and a first blend of pharmaceutical excipients and a second granulation 12 of another pharmaceutical ingredient (in this case, an opioid agonist) and a second blend of pharmaceutical excipients. The pharmaceutical excipients(s) can be selected independently at each occurrence. Likewise, the granulations 11 and 12 can comprise the same or different excipients. The two different granulations can together be filled into a capsule, such as a hard gelatin capsule, or compressed into a tablet core. The granulations are optionally mixed with one or more other pharmaceutical excipients prior to being filled into the capsule or compressed into the tablet core.

The mixed granulation 10 is generally made according to the process of Example 13 and generally by forming a first granulation of an opioid antagonist and a first blend of pharmaceutical excipients, forming a second granulation of an opioid agonist and a second blend of pharmaceutical excipients, and mixing the first and second granulations.

FIG. 5 depicts a pharmaceutical composition of a second granulate 15 containing a mixture 16 of a first granulation 17, a low dose of the opioid antagonist and at least one pharmaceutical excipient, wherein the first granulation comprises a dose of another pharmaceutical ingredient (in this case, an opioid agonist) and at least one pharmaceutical excipient. The pharmaceutical excipients(s) can be selected independently at each occurrence. Plural second granulates can be filled into a capsule, such as a hard gelatin capsule, or compressed into a tablet core. The second granulates are optionally mixed with one or more other pharmaceutical excipients prior to being filled into the capsule or compressed into the tablet core.

The second granulate 15 is made according to the process of Example 14 and generally by forming a first granulation 17 of another pharmaceutical ingredient (in this case, an opioid agonist) and at least one pharmaceutical excipient, and mixing the first granulation with a mixture 16 of the opioid antagonist and at least one pharmaceutical excipient to form a second granulation.

FIG. 6 depicts a pharmaceutical composition 20 of a coated granulation of a mixture of pharmaceutical excipients 23 coated with a binder composition 21 of a binder, the opioid antagonist, another pharmaceutical ingredient (in this case, an opioid agonist) and optionally one or more other pharmaceutical excipients 22. The pharmaceutical excipients(s) can be selected independently at each occurrence. The pharmaceutical composition can be filled into a capsule, such as a hard gelatin capsule, or compressed into a tablet core.

The pharmaceutical composition 20 is made according to the process of Example 15 and generally by preparing a binder composition of a binder, the opioid agonist and the opioid antagonist and coating a mixture of pharmaceutical excipients with the binder composition to form a coated granulation. The coated granulation is then optionally mixed with one or more pharmaceutical excipients to form the pharmaceutical composition 20.

Different methods of treatment will require different dosage strengths and different ratios of agonist to antagonist in a dosage form. The compositions 2 and 10 have the advantage that pharmaceutical composition batches containing substantially different ratios of agonist to antagonist can be easily made simply by varying the amount of each coated nonpareil, or granulation, respectively, included in the pharmaceutical composition without having to reformulate the respective coated nonpareils 1 and 2 or granulations 11 and 12. Likewise the relative amount of each drug within its respective coated nonpareil, or granulation, can be varied to make pharmaceutical compositions of particular drug strengths.

An advantage of a combination dosage form is that the presence of both active pharmaceutical ingredients in a single Unit dose form assures compliance with the desired dose ratio each time a dose is taken. Additional advantages may include reduced complexity, reduced potential for medication errors, and more convenient administration of multiple products concurrently, as well as reduction of the amount being ingested which may accommodate the accompanying symptoms that patients have (for example, nausea, vomiting, inability or exacerbation of pain due to swallowing).

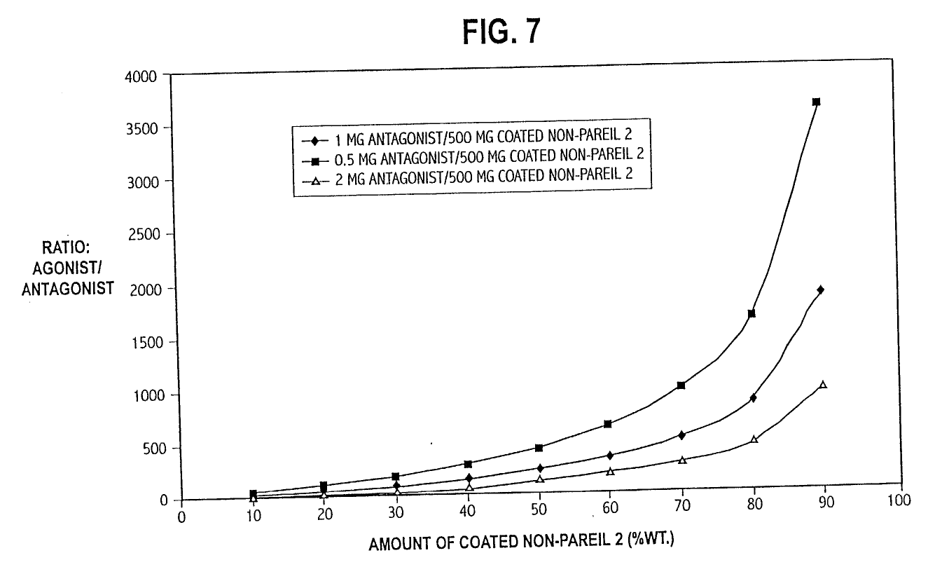

For example, FIG. 7 depicts a graph of the relationship between drug ratio and nonpareil loading into a 500 mg filled capsule. The coated nonpareil 1 can be made to contain 1 mg of opioid antagonist per 500 mg of coated nonpareil 1. Likewise, the coated nonpareil 2 can be made to contain 200 mg of opioid agonist (or another pharmaceutical ingredient) per 500 mg of coated nonpareil 2. Therefore, when a 500 mg capsule is filled up to 90% wt. with nonpareil 2 and 10% wt. with nonpareil 1, the capsule will contain 180 mg of opioid agonist and 0.1 mg of opioid antagonist, and the ratio of agonist to antagonist will be 1800. When a 500 mg capsule is filled up to 50% wt. with nonpareil 2 and 50% wt. with nonpareil 1, however, the capsule will contain 100 mg of opioid agonist and 0.5 mg of opioid antagonist, and the ratio of agonist to antagonist will be 200. On the other hand, when a 500 mg capsule is filled with the same amounts of nonpareil 2 and nonpareil 1, but the nonpareil 1 is made to contain 0.5 mg of antagonist per 500 mg of nonpareil 1, then the capsule will contain 100 mg of opioid agonist and 0.25 mg of opioid antagonist, and the ratio of agonist to antagonist will be 400. The exemplary relationships depicted in FIG. 7 are generally true for the formulations of FIGS. 2 and 4. These relationships will vary according the amount of each drug include in the formulation and the specific drugs used.

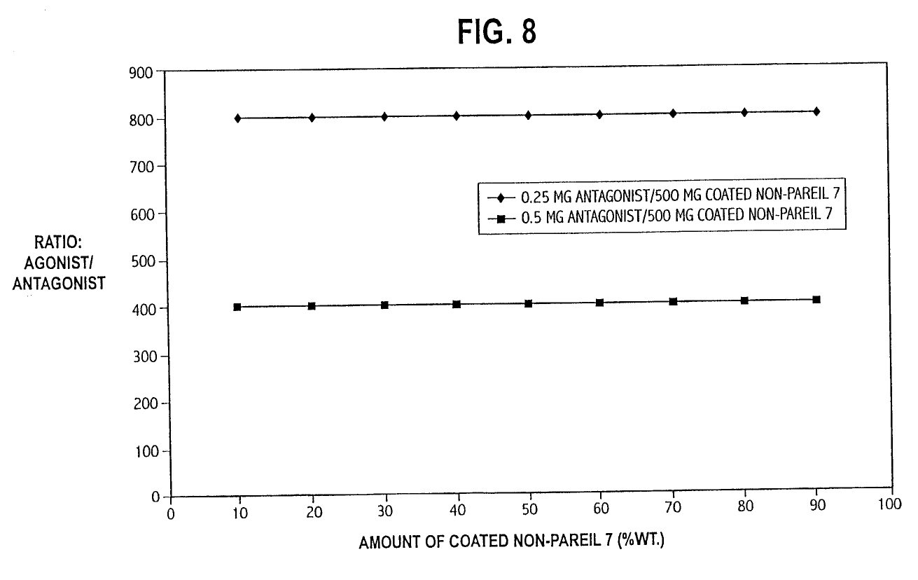

The pharmaceutical compositions of FIGS. 3, 5 and 6, however, are useful for providing a fixed ratio of opioid antagonist to opioid agonist (or another active pharmaceutical ingredient) regardless of the total drug strength of dosage forms containing those pharmaceutical compositions. For example, FIG. 8 depicts a graph of the relationship between the drug ratio and nonpareil loading into a 500 mg filled capsule. The coated nonpareil 7 (shown in FIG. 3) can be made to contain 0.5 mg of opioid antagonist per 500 mg of coated nonpareil 7 and 200 mg of opioid agonist per 500 mg of coated nonpareil 7. Therefore, when a 500 mg capsule is filled 100% wt. with nonpareil 7, the capsule will contain 200 mg of opioid agonist and 0.5 mg of opioid antagonist, and the drug ratio of agonist to antagonist will be 400. When a 500 mg capsule is filled up to 50% wt. with nonpareil 7 and 50% wt. with a pharmaceutical excipient, however, the capsule will contain 100 mg of opioid agonist and 0.25 mg of opioid antagonist, and the ratio of agonist to antagonist will remain 400. If, however, the coated nonpareil 7 is made to contain 0.25 mg of opioid antagonist per 500 mg of coated nonpareil 7 and 200 mg of opioid agonist per 500 mg of coated nonpareil 7 and a 500 mg capsule is filled 100% wt. with nonpareil 7, the capsule will contain 200 mg of opioid agonist and 0.25 mg of opioid antagonist, and the drug ratio of agonist to antagonist will be 800. In other words, decreasing the relative amount of the pharmaceutical compositions of FIGS. 3, 5 and 6 decreases the total drug strength but does not alter the drug ratio.

Another embodiment provides a pharmaceutical composition of a coated first granulate, wherein the granulate comprises the opioid agonist (or another active pharmaceutical ingredient) and a mixture of pharmaceutical excipients and the coating comprises the opioid antagonist and one or more pharmaceutical excipients. This granulate can be made according to the process of Example 16 and is generally made by coating a composition of the opioid agonist and a mixture of pharmaceutical excipients and with a coating composition of the opioid antagonist and one or more pharmaceutical excipients.

The invention also provides a pharmaceutical composition of spray-dried granules of the opioid antagonist, another active pharmaceutical ingredient such as an opioid agonist and at least one pharmaceutical excipient. These granules can be made according to the process of Example 17 and generally by spray-drying a solution of the opioid antagonist, the opioid agonist and at least one pharmaceutical excipient to form spray-dried granules.

The dosage form can also comprise a soft gelatin capsule filled with a suspension of the opioid antagonist in another active pharmaceutical ingredient such as an opioid agonist, and at least one nonaqueous vehicle. This dosage can be made according to the process of Example 18 and generally by filling a soft gelatin capsule with a dispersion consisting essentially of the opioid antagonist, the opioid agonist, and at least one nonaqueous vehicle.

Compositions for inhalation or insufflation include solutions and suspensions in pharmaceutically acceptable, aqueous or organic solvents, or mixtures thereof, and powders are also contemplated. The liquid or solid compositions may contain suitable pharmaceutically acceptable excipients as set out above. The compositions commonly are administered by the oral or nasal respiratory route for local or systemic effect. Compositions in preferably sterile pharmaceutically acceptable solvents may be nebulized by use of inert gases. Nebulized solutions may be breathed directly from a nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

For the treatment of certain conditions it may be desirable to employ an opioid antagonist in conjunction with another pharmacologically active agent. For example, an opioid antagonist according to the present invention may be presented together with another therapeutic agent as a combined preparation for simultaneous, separate, or sequential use. Solid compositions of an opioid antagonist alone may be administered in combination with any one or more other therapeutic agents, including, but not limited to, opioid agonists. Such combined preparations may be, for example, in the form of a twin pack. In general, the currently available dosage forms of the other therapeutic agents for use in such combinations will be suitable.

An opioid antagonist alone, or in combination with another active pharmaceutical ingredient, may be administered to the human subject by known procedures including but not limited to oral, sublingual, intramuscular, subcutaneous, intravenous, intratracheal, transmucosal, or transdermal modes of administration. When a combination of these compounds are administered, they may be administered together in the same composition, or may be administered in separate compositions. If the opioid antagonist and another active pharmaceutical ingredient are administered in separate compositions, they may be administered by similar or different modes of administration, or may be administered simultaneously with one another, or one shortly before or after the other.

Commercial formulations currently used to administer the opioid antagonist or opioid agonist can be modified as described to provide a pharmaceutical composition and formulation according to the invention. Commercial oral dose forms of opioid agonists for human administration include: codeine, dihydrocodeine (e.g. SYNALGOS-DC® from Wyeth-Ayerst Pharmaceuticals), fentanyl (e.g., ACTIQ® from Abbott Laboratories), hydrocodone (e.g., VICODIN® and VICOPROFEN® from Knoll Laboratories; NORCO® from Watson Laboratories; HYCODAN® from Endo Pharmaceuticals; NORCET® from Abara; ANEXSIA®, HYDROCET®, and LORCET-HD® from Mallinckrodt; LORTAB® from UCB Pharma; HY-PHEN® from Ascher; CO-GESIC® from Schwarz Pharma; ALLAY® from Zenith Goldline), hydromorphone (e.g., DILAUDID® from Knoll), levorphanol (e.g., LEVO-DROMORAN® from ICN Pharmaceuticals), meperidine (e.g., DEMEROL® from Sanofi Pharmaceuticals), methadone (e.g., METHADOSE® from Mallinckrodt; and DOLOPHINE® HCl from Roxane Laboratories), morphine (e.g., KADIAN® from Faulding Laboratories; MS CONTIN® from Purdue Frederick; ORAMORPH® SR from Roxane), oxycodone (e.g., PERCOCET® and PERCODAN® from Endo; OXYCET® from Mallinckrodt; OXYCONTIN® from Purdue Frederick; TYLOX® from Ortho-McNeil Pharmaceutical; ROXICODONE®, ROXILOX® and ROXICET® from Roxane), pentazocine (e.g., TALACEN® and TALWIN® from Sanofi Pharmaceuticals), propoxyphene (e.g., DARVOCET-N® and DAVRON® from Eli Lilly & Co.; DOLENE® from Lederle; WYGESIC® from Wyeth-Ayerst), and tramadol (e.g., ULTRAM® from Ortho-NcNeil Pharmaceutical). Commercial liquid dose forms of opioid agonists for human administration include: hydrocodone (e.g., HYDROPHANE® from Halsey), hydromorphone (e.g., DILAUDID® from Knoll), meperidine (e.g., DEMEROL® from Sanofi), methadone (e.g. DOLOPHINE® from Roxane), oxycodone (e.g., HYCOMINE® from Knoll; ROXILOX® from Roxane), and propoxyphene (e.g., DARVON-N® from Eli Lilly). Commercial parenteral dose forms for human administration include: alfentanil (e.g., ALFENTA® from Akorn), buprenorphine (e.g., BUPRENEX® from Reckitt & Colman Pharmaceuticals), butorphanol (e.g., STADOL® from Apothecon), dezocine (e.g., DALGAN® from Astrazeneca), fentanyl, hydromorphone (e.g., DILAUDID-HP® from Knoll), levallorphan (e.g., LORFAN® from Roche), levorphanol (e.g., LEVO-DROMORAN® from ICN), meperidine (e.g., DEMEROL® from Sanofi), methadone (e.g., DOLOPHINE® HCI from Roxane), morphine (e.g. ASTRAMORPH® from Astrazeneca; DURAMORPH® and INFUMORPH® from Elkins-Sinn), oxymorphone (e.g., NUMORPHAN® from Endo), nalburphine (e.g., NUBAIN® from Endo Pharmaceutical), and pentazocine (TALWIN® from Abbott). Commercial transdermal dose forms of opioid agonists for human administration include fentanyl (e.g., DURAGESIC® from Janssen). Commercial suppository dose forms of opioid agonists for human administration include oxymorphone (e.g., NUMORPHAN® from Endo).

The present invention also includes pharmaceutical kits comprising an opioid antagonist together with any other therapeutic agent, including but not limited to, an opioid agonist, where the antagonist is in an amount as specified above. In the kit, the opioid antagonist and the opioid agonist or other therapeutic agent may each be presented in separate containers of any type, for example, bottles or packages (e.g., for capsules, tablets, pills or patches) as compounds, and/or in separate containers as compounds in combination with a pharmaceutically acceptable excipient or carrier. Alternatively, the opioid antagonist and the opioid agonist or other therapeutic agent may be combined together in one or more containers such as bottles or packages with or without an excipient or carrier. Thus, for example, the invention also includes pharmaceutical kits comprising a container of any type, such as a package, bottle, envelope, blister pack, bag, or pouch, syringe, inhaler or tube, consisting essentially of the opioid antagonist and a separate container consisting essentially of the opioid agonist or other therapeutic agent, each container containing, if desired, an excipient or other carrier.

The pharmaceutical compositions may be administered to human subjects/patients in need of such treatment in dosage forms, for example, within the ranges described herein, that will provide acceptable pharmaceutical efficacy. It will be appreciated that the specific dose required for use in any particular application will vary from patient to patient, not only with the particular compound or composition selected, but also with the route of administration, the nature of the condition being treated, the age, condition, pain tolerance, and other idiosyncrasies of the patient, concurrent medication or special diets then being followed by the patient, and other factors which those skilled in the art will recognize, with the appropriate dosage ultimately being at the discretion of the attendant physician.

In preferred embodiments, when the opioid antagonist is administered alone, the amount of the opioid antagonist administered is an amount effective to enhance or maintain the analgesic potency of the opioid agonist and/or attenuate or maintain the adverse side effects of the opioid agonist and/or attenuate tolerance, withdrawal, dependence and/or addiction. This amount is readily determinable by one skilled in the art.

The present invention is described in the following examples, which are set forth to aid in the understanding of the invention and should not be construed to limit in any way the invention as defined in the claims which follow thereafter. Pharmaceutical active and inactive ingredients used in the preparation of example formulations were compendial in the USP/NF, when there was an existing monograph.

### EXAMPLE 1

The drug product is manufactured to contain multiple active components that include the opioid antagonist in one unit dose (e.g., a single capsule or tablets or pill or patch). Alternatively, the drug product is manufactured to contain only one active component that is the opioid antagonist (e.g., naltrexone). Naltrexone hydrochloride (naltrexone) were manufactured as described herein and administered as a separate capsule in dosage forms according to the invention.

The description, structure and physical/chemical characteristics of the drug substance Naltrexone Hydrochloride, USP is as follows:

Generic Name: Naltrexone Hydrochloride

Chemical Name: 17-cyclopropylmethyl-4,5α-epoxy-3,14-dihydroxymorphinan-6-one hydrochloride

Molecular Formula: C₂₀H₂₃NO₄.HCl

Molecular Weight: 377.86

(See, e.g., USP description for details on the physical and chemical properties of naltrexone hydrochloride.)

Naltrexone Capsules

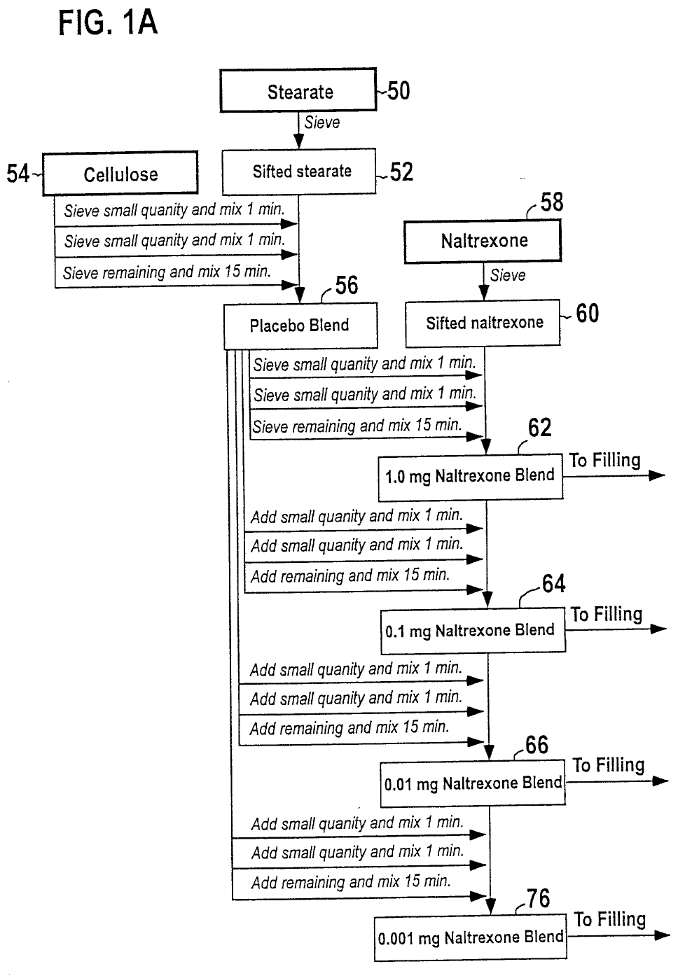

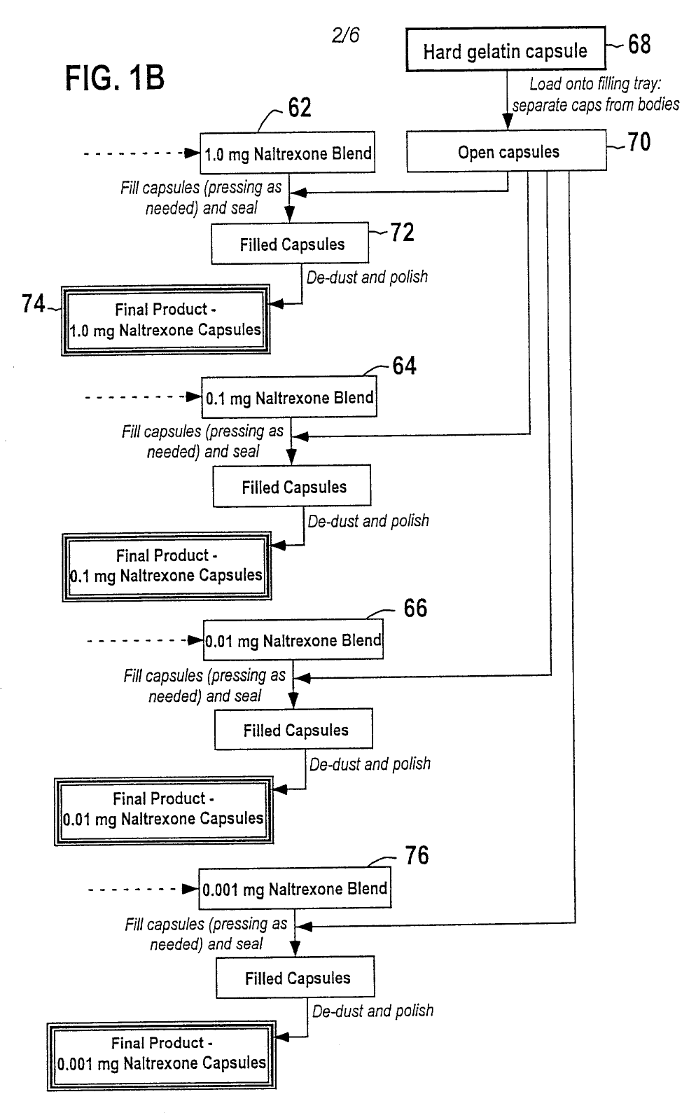

Naltrexone capsules were manufactured in representative concentrations of 0.01 mg, 0.1 mg and 1.0 mg naltrexone HCl by the process illustrated in FIGS. 1A-1B and described below.

| | | |
|---|---|---|
| Components | Per Capsule | Per Batch¹ |

0.01 mg Naltrexone Capsules

| | | | | |
|---|---|---|---|---|
| Naltrexone Hydrochloride, USP | 0.01 | mg | 0.013 | g |
| grade² | | | | |
| Microcrystalline cellulose, NF | 346.24 | mg | 432.80 | g |
| grade³ | | | | |
| Magnesium stearate, NF grade⁴ | 1.75 | mg | 2.19 | g |
| Hard gelatin capsule shells | 1 | | 1250 | |
| (opaque white/white, size 0), NF | | | | |
| grade⁵ | | | | |

0.1 mg Naltrexone Capsules

| | | | | |
|---|---|---|---|---|
| Naltrexone Hydrochloride, USP | 0.10 | mg | 0.13 | g |
| grade² | | | | |
| Microcrystalline cellulose, NF | 346.15 | mg | 432.69 | g |
| grade³ | | | | |
| Magnesium stearate, NF grade⁴ | 1.75 | mg | 2.19 | g |
| Hard gelatin capsule shells | 1 | | 1250 | |
| (opaque white/white, size 0), NF | | | | |
| grade⁵ | | | | |

1.0 mg Naltrexone Capsules

| | | | | |
|---|---|---|---|---|
| Naltrexone Hydrochloride, USP | 1.00 | mg | 1.25 | g |
| grade² | | | | |
| Microcrystalline cellulose, NF | 345.25 | mg | 431.57 | g |
| grade³ | | | | |
| Magnesium stearate, NF grade⁴ | 1.75 | mg | 2.18 | g |
| Hard gelatin capsule shells | 1 | | 1250 | |
| (opaque white/white, size 0), NF | | | | |
| grade⁵ | | | | |

Placebo Capsules

| | | | | |
|---|---|---|---|---|
| Microcrystalline cellulose, NF | 346.25 | mg | 415.5 | g |
| grade² | | | | |
| Magnesium stearate, NF grade³ | 1.75 | mg | 2.1 | g |
| Hard gelatin capsule shells | 1 | | 1200 | |
| (opaque white/white, size 0), NF | | | | |
| grade⁴ | | | | |

¹Amounts listed are for a 1250 capsule batch. Other batch sizes may be used with equivalent formula, using similar equipment and processing.
²Supplied in 10 g. bottles by Mallinckrodt Chemicals, Inc., P.O. Box 5439, St. Louis, MO 63147.
³Supplied as Avicel ® PH102 by FMC Corporation, Pharmaceutical Division, 1735 Market St. Philadelphia, PA 19103.
⁴Supplied by Whittaker, Clark & Danield, Inc. 1000 Coolidge St., South Plainfield, NJ 07080
⁵Supplied by Capsugel ®, Division of Wamer-Lambert Co., 535 N. Emerald Rd., Greenwood, SC 29646
¹Amounts listed are for a 1200 capsule batch. Other batch sizes may be used with equivalent formula, using similar equipment and processing.
²Supplied as Avicel ® PH102 by FMC Corporation, Pharmaceutical Division, 1735 Market St. Philadelphia, PA 19103.
³Supplied by Whittaker, Clark & Danield, Inc. 1000 Coolidge St., South Plainfield, NJ 07080

The manufacturing schematic of FIGS. 1A and 1B was employed to manufacture naltrexone capsules useful for human administration. A description of the steps of the manufacturing process for naltrexone and placebo capsules follows:

For three selected concentrations (0.01 mg, 0.1 mg, 1.0 mg) of naltrexone capsules, a series of blends were made and combined to create the specified concentrations for filling. Additional blends were made and combined to create concentrations less than 0.01 mg, such as 0.001 mg. Further blends can be made at less than 0.001 mg, including 0.0001 mg or less.

Placebo Blend

A placebo blend was made according to the following process.

Referring first to FIG. 1A, magnesium stearate 50 was placed through a clean, 60 mesh stainless sieve and the sifted stearate 52 was collected directly into a blender bowl. A small portion of microcrystalline cellulose 54 was placed through the same sieve and collected over the sifted stearate 52, then mixed well for at least 1 minute. This was repeated with another small portion of cellulose. The remaining cellulose was placed through the sieve and mixed well for 15 minutes. The resulting blend 56 was checked for visible contaminants.

1.0 mg Naltrexone Blend

The entire quantity of naltrexone 58 was placed through a clean, 60 mesh stainless sieve and the sifted naltrexone 60 was collected directly into a blender bowl. A small portion of the placebo blend 56 was placed through the same sieve and collected over the naltrexone, then mixed well for at least 1 minute. This was repeated with another small portion of placebo blend. The remaining placebo blend was placed through the sieve and mixed well for 15 minutes. The result was the 1.0 mg naltrexone blend 62. The blend was checked for visible contaminants. The blend 62 was either further diluted as described in the next paragraph or used to fill capsules (as described further below) if a 1.0 mg dosage form was desired as the final product.

0.1 mg Naltrexone Blend

A specified amount of the 1.0 mg naltrexone blend 62, made as described above, was placed into a blender bowl. A small portion of placebo blend 56 was placed over the 1.0 mg naltrexone blend 62, then mixed well for at least 1 minute. This was repeated with another small portion of the placebo blend 56. The remaining portion of the placebo blend 56 required to provide a 10:1 dilution was added and mixed well for 15 minutes. The result was the 0.1 mg naltrexone blend 64. The blend 64 was checked for visible contaminants. The blend 64 was either further diluted as described in the next paragraph or used to fill capsules (as described further below) if a 0.1 mg dosage was desired as the final product.

0.01 mg Naltrexone Blend

A specified amount of the 0.1 mg naltrexone blend 64, made as described above, was placed into a blender bowl. A small portion of placebo blend 56 was placed over the 0.1 mg naltrexone blend 64, then mixed well for at least 1 minute. This was repeated with another small portion of the placebo blend 56. The remaining portion of the placebo blend 56 required to provide a 10:1 dilution were added and mixed well for 15 minutes. The result was the 0.01 naltrexone blend 66. The blend 66 was checked for visible contaminants.

Analogous dilution has been carried out with a specified amount of the 0.01 mg naltrexone blend to achieve a 0.001 mg naltrexone blend 76 (as shown in FIGS. 1A and 1B). Further dilutions are also contemplated, if a smaller dose is desired.

Filling Capsules

With reference to FIG. 1B (a continuation of FIG. 1A), the following filling procedure was used to put each naltrexone dosage form 62, 64, and 66 into capsules. 1250 empty capsule shells 68 were loaded onto filling trays. The caps were separated from the bodies—shown as step 70.

The amount of the 1.0 mg naltrexone blend 62 needed to fill 1250 capsules (including 1% overage) was determined. This amount of specified concentration naltrexone blend was weighed and transferred to the capsule machine. The capsules 70 were filled with the entire quantity of naltrexone blend, applying three pressings for each filling operation. The capsules were sealed to form filled capsules 72. The capsules 72 were de-dusted and polished. The filled capsules 72 were tested for weight variation, and only those capsules 72 within specified range were accepted as the final product—1.0 mg naltrexone capsules 74. The capsules 74 were collected into secure, labeled, double polyethylene bags.

In a similar fashion, the 0.1 mg naltrexone blend 64 and the 0.01 mg naltrexone blend were put into capsules and finished, again as illustrated in FIG. 1B.

A process of serial dilution and blending was used to manufacture lower concentration strength naltrexone capsules. Additionally, reduction in the capsule fill weight has been used to accomplish proportional reductions in naltrexone capsule strength as necessary, for example, to manufacture 0.0001 mg naltrexone capsules.

To form placebos, the placebo blend 56 was used in place of the naltrexone blend 62 to fill capsules, which were again finished in the same manner.

### EXAMPLE 2

This example demonstrates the clinical use and evaluation of solid oral dosage forms, including some of the dosage forms of Example 1. Pharmaceutical compositions and dosage forms of naltrexone prepared according to the procedure stated in Example 1 were administered in human clinical trials with morphine, tramadol or hydrocadone/acetaminophen.

A study of morphine alone and in combination with naltrexone is described in Example 1 of U.S. Application No. 60/202,265, filed May 5, 2000, incorporated by reference herein. A summary of exemplary study results follows.

The clinical study was designed to compare the analgesic activity of three different doses of naltrexone (NTX) in combination with morphine sulfate (hereafter called morphine or MS) 60 mg, versus MS 60 mg alone. The test subjects had moderate to severe postsurgical dental pain. The test products were MS 60 mg with naltrexone (NTX) 1 mg, MS 60 mg with NTX 0.1 mg, and MS 60 mg with NTX 0.01 mg. A single oral dose of one of the treatments was administered when the subject was suffering moderate to severe postoperative pain. The Study Population was 201 male and female outpatients with moderate to severe pain following extraction of two or more impacted third molars.

For the data analysis, certain pain parameters were computed as follows. The extent to which pain changes at each time point was measured by pain relief scores (PR, with 0=none, 1=a little, 2=some, 3=a lot, 4=complete), and pain intensity difference scores (PID, the difference between baseline and the current time, with the pain intensity scale consisting of 0=none, 1=mild, 2=moderate, 3=severe). The extent to which pain changes over the entire test period was measured by the total pain relief score (TOTPAR-8), sum of pain intensity differences (SPID-8), maximum pain relief score (MAXPAR), peak pain intensity difference (PEAKPID), and global evaluation (0=poor, 1=fair, 2=good, 3=very good, 4=excellent). TOTPAR-8 and SPID-8 are defined as the sum of PR and PID, respectively, for the entire 8-hour observation period, weighted by the time difference between adjacent points (i.e., area under the curve using the trapezoidal rule). MAXPAR and PEAKPID are defined as the maximum of PR and PID, respectively. The efficacy and safety evaluations included global pain evaluation, time to rescue, percent of patients remedicating with rescue medication, time to onset of meaningful pain relief, time to onset of first perceptible pain relief and visual analog scale (VAS), and adverse event assessments.

The placebo treatment group had the lowest mean Total Pain Relief scores. All 4 of the active treatment groups exhibited mean Total Pain Relief scores that were numerically higher than placebo. The combination treatments had a reverse dose-response relation in the mean Total Pain Relief scores, i.e., the highest dose of NTX had the lowest mean Total Pain Relief scores and the lowest dose of NTX had the highest mean Total Pain Relief scores. This pattern (low-dose (0.01 mg NTX)>mid-dose (1.0 mg NTX) was observed for all pain relief variables throughout the study. The mean Total Pain Relief scores for the 0.01-mg NTX and 0.1-mg NTX combination treatments were higher than that for the MS alone treatment, whereas the 1.0-mg NTX combination treatment mean was comparable to or lower than that for the MS alone treatment.

The placebo treatment had the lowest mean 4-hour Sum of Pain Intensity Differences scores. All 4 of the active treatment groups exhibited improved profiles in mean Sum of Pain Intensity Differences relative to placebo. The mean Sum of Pain Intensity Differences scores for the 0.01-mg NTX and 0.1-mg NTX combination treatments were higher than that for the MS alone treatment, whereas the 1.0-mg NTX combination treatment was comparable to that for the MS alone treatment. The patterns of the 6-hour and 8-hour Sum of Pain Intensity Differences scores were similar to those at 4 hours.

The median time to onset of meaningful pain relief was shortest in the 0.01-mg NTX (low-dose) combination treatment group. The placebo treatment had the lower number of subjects who reached meaningful pain relief.

The majority of adverse side effects reported were categorized as digestive (nausea or vomiting) or nervous system (dizziness or somnolence).

A study of tramadol alone and in combination with naltrexone is described in Example 10 of U.S. application Ser. Nos. 09/756,331, filed Jan. 8, 2001, which is a continuation of 09/566,071, filed May 5, 2000 and PCT/US00/12493 [WO/00 67739] filed May 5, 2000, the entire disclosures of which are hereby incorporated by reference. A summary of exemplary study results follows.

In this study in human subjects with pain, tramadol hydrochloride (tramadol) was administered alone or in combination with various amounts (doses) of an opioid antagonist, naltrexone. In this study, one objective was to determine whether an opioid antagonist such as naltrexone hydrochloride (hereafter referred to in this example as naltrexone or NTX) enhances the analgesic properties of tramadol hydrochloride (hereafter referred to in this example as tramadol or T) in human subjects/patients with pain following dental surgery. An additional objective was to evaluate whether an opioid antagonist such as NTX attenuated (e.g., reduced, blocked or prevented) tramadol's adverse side effects in humans.

Human subjects were randomized into one of the following five treatment groups:

Group 1: T (50 mg) with NTX (1 mg)

Group 2: T (50 mg) with NTX (0.1 mg)

Group 3: T (50 mg) with NTX (0.01 mg)

Group 4: T (50 mg) with Placebo

Group 5: Placebo with Placebo

All subjects with moderate to severe pain received one dose of study medication. Subjects received two capsules to take by mouth, one tramadol or placebo, the other naltrexone or placebo.

A pain assessment was performed pre-treatment. Following the dental surgery, the subject's pain level was assessed by a trained observer. The subject reported the initial pain intensity by both (1) verbalizing one pain category (0=none, 1=mild, 2=moderate or 3=severe), and (2) using a Visual Analog Scale (VAS) of 0-100 mm where 0=no pain and 100=worst pain imaginable, by placing a single slash on the scale. A pain assessment was also performed post-treatment.

The efficacy and safety evaluations included pain intensity, pain relief, global pain evaluation, evaluation of time to meaningful pain relief (stop watch), visual scale analog (VAS), and adverse event assessments. For the data analysis, certain pain parameters were computed as generally described above.

The placebo treatment group had the lowest mean 4-hour Total Pain Relief scores. All 4 of the active treatment groups exhibited mean 4-hour Total Pain Relief scores that were numerically higher than placebo. The combination treatments had a reverse dose-response relation in the mean 4-hour Total Pain Relief scores, i.e., the highest dose of NTX had the lowest mean 4-hour Total Pain Relief scores and the lowest dose of NTX had the highest mean 4-hour Total Pain Relief scores. The mean 4-hour Total Pain Relief scores for the 0.01-mg NTX and 0.1-mg NTX combination treatments were higher than that for the T alone treatment, whereas the 1.0-mg NTX combination treatment mean was lower than that for the T alone treatment.

The placebo treatment had the lowest mean 4-hour Sum of Pain Intensity Differences scores. All 4 of the active treatment groups exhibited improved profiles in mean 4-hour Sum of Pain Intensity Differences relative to placebo. The mean 4-hour Sum of Pain Intensity Differences scores for the 0.01-mg NTX and 0.1-mg NTX combination treatments were higher than that for the T alone treatment, whereas the 1.0-mg NTX combination treatment was lower than that for the T alone treatment. The patterns of the 6-hour and 8-hour Sum of Pain Intensity Differences scores were similar to those at 4 hours.

The 4, 6, and 8 hour Visual Analog Scale Sum of Pain Intensity Differences results were as follows. The placebo treatment had the lowest mean 4-hour VAS-Sum of Pain Intensity Differences. The 4 active treatment groups exhibited mean VAS-Sum of Pain Intensity Differences scores that were higher than that for the placebo. The mean 4-hour VAS-Sum of Pain Intensity Differences for the 3 NTX combination treatments was higher than that for T alone. The profiles of 6-hour and 8-hour VAS-Sum of Pain Intensity Differences scores were similar to those at 4 hours.

The placebo treatment had the lowest number of subjects who reached meaningful pain relief. In addition, all the combination treatment groups had higher numbers of subjects reaching meaningful pain relief than did the group that received T alone.

Whereas the hourly pain relief scores for the placebo treatment were generally flat, those for the active treatment groups were generally improving over time. There was separation between the placebo and the active treatment groups that continued throughout the 8-hour study period.

The majority of adverse events reported were categorized as gastrointestinal disorders (nausea or vomiting) or nervous system disorders (dizziness, headache or sedation).

A study of morphine alone and in combination with naltrexone is described in Example 2 of U.S. Application Ser. No. 60/245,110, filed Nov. 1, 2000, incorporated by reference herein. A summary of exemplary study results follows.

In a dose ranging study, doses of morphine sulfate (60 mg) in combination with naltrexone hydrochloride (0.1 mg, 0.01 mg, or 0.001 mg) were administered for moderate to severe pain in patients following dental surgery. This study was performed to investigate the analgesic efficacy (onset, peak, duration, and total effect) of morphine alone, naltrexone alone, three different doses of naltrexone in combination with morphine and placebo.

The 300 subject study was designed with six treatment groups: A) placebo (50 pts); B) morphine 60 mg (50 pts); C) naltrexone 0.01 mg (50 pts); D) morphine 60 mg and naltrexone 0.1 mg (50 pts); E) morphine 60 mg and naltrexone 0.01 mg (50 pts); F) morphine 60 mg and naltrexone 0.001 mg (50 pts). In this study, in the treatment of moderate to severe pain following extraction of 3 or 4 full or partial bony impacted third molars, a single oral dose of one of the treatments was administered when the patient was suffering moderate to severe postoperative pain. The observation period for efficacy was 8 hours post treatment, and for safety was 24 hours post treatment.

The efficacy and safety evaluations included pain intensity, pain relief, global pain evaluation, evaluation of time to meaningful pain relief (stopwatch), visual analog scale (VAS), and adverse event assessments. For the data analysis, certain pain parameters were computed as generally described above.

The 0.01 mg NTX alone and placebo treatment groups had the lowest mean 4 hour Sum of Pain Intensity Difference (SPID) scores. All 4 of the active treatment groups with MS alone or in combination with NTX exhibited improved profiles in mean SPID relative to NTX alone or placebo. The mean 4 hour SPID scores for the 0.01 mg NTX and 0.1 mg NTX combination treatments were higher than that for the MS alone treatment, whereas the 0.001 mg NTX combination treatment was comparable to that for the MS alone treatment. The patterns of the 6 hour and 8 hour SPID scores were similar to those at 4 hours.

The 0.01 mg NTX alone and placebo treatment groups had the lowest mean Total Pain Relief scores. All 4 of the active treatment groups exhibited mean Total Pain Relief scores that were numerically higher than the 0.01 mg NTX alone and placebo treatment groups. The combination treatments had a dose-response relation in the mean Total Pain Relief scores, i.e., the highest dose of NTX had the highest mean Total Pain Relief scores and the lowest dose of NTX had the lowest mean Total Pain Relief scores. This pattern (high-dose (0.1 mg NTX)>mid-dose (0.01 mg NTX)>low-dose (0.001 mg NTX) was generally observed for pain relief variables throughout the study. The mean Total Pain Relief score for the 0.01 mg NTX and the 0.1 NTX combination treatment groups were higher than that for the MS alone treatment, whereas the 0.001 mg NTX combination treatment mean was comparable to or lower than that for the MS alone treatment.

The NTX alone and placebo treatment groups had the highest number of subjects who had “poor” global evaluation scores. The profiles of the global evaluations scores are based on subjects' evaluations.

The median time to onset of meaningful pain relief was shortest in the 0.1 mg NTX combination treatment group.

The median time to onset of analgesia was shortest in the 0.1 mg NTX combination treatment group.

The baseline pain intensity scores and visual analog scale scores were generally comparable across treatment groups.

The majority of adverse events reported were categorized as digestive (nausea or vomiting) or nervous system (dizziness or somnolence).

A study of hydrocodone with acetaminophen (instead of morphine) alone and in combination with naltrexone is described in Example 3 of U.S. Application Ser. No. 60/245,110, filed Nov. 1, 2000, incorporated by reference herein. A summary of exemplary study results follows.

In a dose ranging study, doses of hydrocodone (5 mg) with acetaminophen (500 mg) and naltrexone (0.1 mg, 0.01 mg, and 0.001 mg) were administered for moderate to severe pain following dental surgery. The study was performed to investigate the analgesic efficacy of hydrocodone with acetaminophen alone, four different doses of naltrexone in combination with hydrocodone/acetaminophen, and placebo.

The 300 subject study was designed with six treatment groups: A) placebo (50 pts); B) HC 5 mg/APAP 500 mg and placebo (50 pts); C) HC 5 mg/APAP 500 mg and NTX 1.0 mg (50 pts); D) HC 5 mg/APAP 500 mg and NTX 0.1 mg (50 pts); E) HC 5 mg/APAP 500 mg and NTX 0.01 mg (50 pts); F) HC 5 mg/APAP 500 mg and NTX 0.001 mg (50 pts). In this study, in the treatment of moderate to severe pain following extraction of 3 or 4 full or partial bony impacted third molars, a single oral dose of one of the treatments was administered when the patient was suffering moderate to severe postoperative pain. The observation period for efficacy was 8 hours post treatment and for safety was 24 hours post treatment.

The efficacy and safety evaluations included pain intensity, pain relief, global pain evaluation, evaluation of time to meaningful pain relief (stopwatch), visual analog scale and adverse event assessments. For the data analysis, certain pain parameters were computed as generally described above.

The placebo treatment group had the lowest mean 4 hour Sum of Pain Intensity Difference (SPID) scores. All 5 of the active treatment groups with HC/APAP alone or in combination with NTX exhibited improved profiles in mean SPID relative to placebo. The mean 4 hour SPID score for the 0.001 mg NTX combination treatment was higher than that for the HC/APAP alone treatment, whereas the other NTX combination treatments were comparable to or lower than that for the HC/APAP alone treatment. The patterns of the 6 hour and 8 hour SPID scores were similar to those at 4 hours.

The placebo treatment group had the lowest mean Total Pain Relief scores. All 5 of the active treatment groups with HC/APAP alone or in combination with NTX exhibited mean Total Pain Relief scores that were numerically higher than placebo. The mean Total Pain Relief score for the 0.001 mg NTX combination treatment was higher than that for the HC/APAP alone treatment, whereas the other NTX combination treatment means were comparable to or lower than that for the HC/APAP alone treatment.

The placebo treatment group had the highest number of subjects who had “poor” global evaluation scores. The 0.001 mg NTX combination treatment group had the highest number of subjects with a total of “excellent”, “very good” and “good” global evaluation scores. The profiles of the global evaluation scores are based on subjects' evaluations.

The median time to onset of meaningful pain relief was shortest in the 0.001 mg NTX (lowest-dose) combination treatment group. The placebo and the 0.01 mg NTX combination treatment groups had the lowest number of subjects who reached meaningful pain relief.

The baseline pain intensity scores and visual analog scale scores were generally comparable across treatment groups.

The majority of adverse events reported were categorized as digestive (nausea or vomiting) or nervous system (dizziness or sedation).

Another study of morphine alone and in combination with naltrexone was conducted. In this dose ranging study, doses of morphine sulfate (30 mg, 60 mg, and 90 mg) in combination with naltrexone hydrochloride (0.1 mg) were administered for moderate to severe pain in male patients following dental surgery. This study was performed to investigate the efficacy of combinations of different doses of morphine with naltrexone 0.1 mg, relative to placebo and relative to morphine alone, to evaluate the dose-response effects of morphine when administered alone and when coadministered with naltrexone 0.1 mg, and to examine the consistency of effect of naltrexone 0.1 mg across different doses of morphine, when coadministered with morphine.

The 210 subject study was designed with seven treatment groups: A) placebo (30 pts); B) morphine 30 mg (30 pts); C) morphine 60 mg (30 pts); D) morphine 90 mg (30 pts); E) morphine 30 mg and naltrexone 0.1 mg (30 pts); F) morphine 60 mg and naltrexone 0.1 mg (30 pts); G) morphine 90 mg and naltrexone 0.1 mg (30 pts). In this study, in the treatment of moderate to severe pain following extraction of 3 or 4 full or partial bony impacted third molars, a single oral dose of one of the treatments was administered when the patient was suffering moderate to severe postoperative pain. The observation period for efficacy was 8 hours post treatment and for safety was 24 hours post treatment.

The efficacy and safety evaluations included PID, SPID-4, SPID-6 and SPID-8, PEAKPID, VAS-PID at each assessment, VAS-SPID-4, -6, and -8, PEAK-VAS-PID, pain relief (PR) score, TOTPAR-4, -6, and -8, MAXPAR, global evaluation, time to onset of analgesia, time to re-medication, percent of patients re-medicating by 4, 8, 24 hours, and adverse event assessments. For the data analysis, certain pain parameters were computed as generally described above.

The mean PID scores for the placebo treatment group were generally flat while the mean PID scores generally improved over time for the active treatment groups (30 mg MS, 60 mg MS and 90 mg MS alone or in combination with 0.1 mg NTX). The mean scores for the morphine alone and morphine/naltrexone combination treatment groups were higher than the mean PID scores for the placebo group at each hourly assessment time from 1-8 hours. Highest pain relief as measured by PID scores was observed for the 90 mg MS/0.1 mg NTX combination treatment group.

The placebo treatment group had the lowest mean 4 hour Sum of Pain Intensity Difference (SPID) scores. All 6 of the active treatment groups with 30 mg, 60 mg or 90 mg MS alone or in combination with 0.1 mg NTX exhibited improved profiles in mean SPID relative to placebo. The mean 4 hour SPID score for the 90 mg MS/0.1 mg NTX combination treatment was the highest among all treatment groups. The mean SPID scores for the 30 mg, 60 mg and 90 mg MS alone treatment groups were comparable. In contrast, the mean SPID scores for the 30 mg MS/0.1 mg NTX, 60 mg MS/0.1 mg NTX and 90 mg MS/0.1 mg NTX combination treatment groups demonstrated a dose response, with the 90 mg MS/0.1 mg NTX combination treatment group having the highest mean SPID-4 scores, and the 30 mg MS/0.1 NTX combination treatment group having the lowest mean SPID-4 scores of the combination treatment groups.

The placebo treatment group had the lowest mean Total Pain Relief scores. All 6 of the active treatment groups with 30 mg, 60 mg or 90 mg MS alone or in combination with 0.1 mg NTX exhibited mean Total Pain Relief scores that were numerically higher than placebo. The mean Total Pain Relief score for the 90 mg MS/0.1 mg NTX combination treatment was the highest among all treatment groups. The mean Total Pain Relief scores for the 30 mg, 60 mg and 90 mg MS alone treatment groups were comparable. In contrast, the mean Total Pain Relief scores for the 30 mg MS/0.1 mg NTX, 60 mg MS/0.1 mg NTX and 90 mg/MS 0.1 mg NTX combination treatment groups demonstrated a dose response, with the 90 mg MS/0.1 mg NTX combination treatment group having the highest mean Total Pain Relief scores, and the 30 mg MS/0.1 NTX combination treatment group having the lowest mean Total Pain Relief scores of the combination treatment groups.

The mean PEAKPID scores varied among treatment groups, and were greater for all 6 active treatment groups compared to the placebo group. Compared to all other groups, the mean PEAKPID scores were highest for the 90 mg MS/0.1 mg NTX combination treatment group.

The mean pain relief score for the placebo treatment was less than those for the active treatment groups (30 mg, 60 mg, 90 mg MS alone or in combination with 0.1 mg NTX) which improved over time. There was separation between the placebo and the active treatment groups that continued throughout the 8 hour study period. Highest pain relief scores were observed for the 90 mg MS/0.1 mg NTX combination group.

The mean MAXPAR scores varied among treatment groups. The mean MAXPAR score was highest for the 90 mg MS/0.1 mg NTX combination treatment group compared to all other groups. The mean scores for all 6 active treatment groups were greater than the mean score for the placebo group.

The placebo treatment group had the highest number of subjects who had “poor” global evaluation scores. The 90 mg MS/0.1 mg NTX combination treatment group had the highest number of subjects with a total of “excellent”, “very good” and “good” global evaluation scores. The profiles of the global evaluation scores are based on subjects' evaluations.

The median time to onset of analgesia was shortest in the 90 mg MS/0.1 mg NTX combination treatment group.

The placebo group had the shortest median time to remedication and the 90 mg MS/0.1 mg NTX combination treatment group had the longest median time to remedication. More than 70% of subjects at 4 hours in the 90 mg MS/0.1 mg NTX combination group and more than 60% of subjects in the same combination group at 8 hours did not require rescue medication.

The baseline pain intensity scores and visual analog scale scores were generally comparable across treatment groups.

The majority of adverse events reported were categorized as digestive (nausea or vomiting) or nervous system (dizziness or somnolence).

### EXAMPLE 3

This example describes the preparation of a dosage form comprising naltrexone hydrochloride (referred to as “naltrexone” or “NTX” in these examples) which was used in clinical evaluations. This example also describes the measurement of the dissolution of that dosage form.

The dosage form for the clinical evaluation and the dissolution tests of this example were provided by preparing hard gelatin capsules comprising an opioid antagonist and excipients according to Example 1. The capsules contained naltrexone HCl and two and pharmaceutical excipients. The tables below indicate the qualitative composition of the NTX capsules.

Components of Naltrexone HCl Capsules

| Component | Description |
|---|---|
| Naltrexone HCl, USP | Active Pharmaceutical Ingredient |
| Microcrystalline Cellulose, NF | Disintegrant/Filler |
| Magnesium Stearate, NF | Lubricant |
| Hard Gelatin Capsule | Unit Dose Form |

The NTX capsules were prepared by filling the empty capsule shells with a powder blend of NTX, microcrystalline cellulose, and magnesium stearate. The fill weight was constant for each strength of NTX capsules produced.

Unless otherwise specified, and subject to the particular conditions and parameters disclosed for the particular examples, the dissolution tests in Example 3 and the other examples were performed using the dissolution test in U.S. Pharmacopeia 24 (2000) Physical Test <711> (which is incorporated herein by reference), employing Apparatus 2 (Paddle Stirring Element).

Dissolution testing was performed on two capsules per dissolution vessel in a media of 500 ml of 0.1 N hydrochloric acid (HCl). The testing media temperature was maintained at about 37.0° C. (+/−0.5° C.), and the paddle speed was 50 rpm. Sample aliquots of the testing media were withdrawn at various time points, and the quantity of naltrexone (and/or another ingredient whose dissolution was to be tested) was determined. The release of the tested ingredient is reported as percentage released versus time.

The table below summarizes the results obtained for the dissolution tests on the NTX capsules. These results are the mean values of six tests.

Dissolution of Naltrexone Hydrochloride from Example 3

| Time (min) | NTX Released (%) |
|---|---|
| 0 | 0 |
| 5 | 94.2 |
| 10 | 91.4 |

The dosage form provided for rapid dissolution of over 90% of the theoretical amount of naltrexone with about 5-10 minutes.

### EXAMPLE 4

This example describes the preparation of a dosage form comprising morphine sulfate pentahydrate (MS) for clinical evaluation. This example also describes the measurement of the dissolution of that dosage form. The dosage form for clinical evaluation and for the dissolution tests of this example were hard gelatin capsules. The MS capsules were prepared by placing 1 to 4 tablets of a commercially available 15 mg MS immediate release tablet into a hard gelatin capsule. A mixture of microcrystalline cellulose and magnesium stearate was then added to bring the capsule to volume and facilitate processing. The table below indicates the qualitative composition of the MS capsules.

Components of Morphine Sulfate Capsules

| Component | Description |
|---|---|
| Morphine Sulfate, Pentahydrate | Active Pharmaceutical Ingredient |
| (as a 15 mg Commercial MS Tablet) | |
| 1-4 tablets | |
| Microcrystalline Cellulose, NF | Disintegrant/Filler |
| Magnesium Stearate, NF | Lubricant |
| Hard Gelatin Capsule | Unit Dose Form |

The in vitro dissolution rates in this example were determined using the Apparatus 2 (Paddle Stirring Element) in U.S. Pharmacopeia 24 (2000), using the same conditions and parameters as described in Example 3.

For dissolution testing, one 60 mg MS capsule was placed in the testing media. The table below summarizes the results obtained for the dissolution tests on the MS capsules. These results are the mean values of six tests.

Dissolution of Morphine Sulfate, Pentahydrate from Example 4

| Time (min) | MS Released (%) |
|---|---|
| 0 | 0 |
| 5 | 78.2 |
| 10 | 89.4 |

These dissolution results show that this dosage form provides immediate release of morphine sulfate pentahydrate.

### EXAMPLE 5

This example describes the preparation of a dosage form comprising oxycodone hydrochloride. This example also describes the measurement of the dissolution of that dosage form. The following general procedure was used to prepare capsules containing different strengths of oxycodone hydrochloride (referred to in this example as oxycodone) supplied as Roxicodone® (Roxane Laboratories, Inc.). Roxicodone® tablets are available in 5.0 mg (oxycodone hydrochloride) strength tablets. Each Roxicodone® tablet is claimed to have a theoretical drug content of 5.0 mg oxycodone hydrochloride, although the actual amount may vary within an acceptable range, for example by about 10%.

A 5.0 mg oxycodone hydrochloride capsule was made by mixing microcrystalline cellulose (229.2 mg) with magnesium stearate (1.2 mg) to form a blend. The blend and one Roxicodone® tablet were loaded separately into the body component of a hard gelatin capsule shell. The capsule was then closed with the cap component of the shell. The filled capsule weighed approximately 426.0 mg.

A 15.0 mg oxycodone hydrochloride capsule was made by mixing microcrystalline cellulose (135.5 mg) with magnesium stearate (0.7 mg) to form a blend. The blend and three Roxicodone® tablets were loaded separately into the body component half of a hard gelatin capsule shell. The capsule was then closed with the cap component of the shell. The filled capsule weighed approximately 533.0 mg.

The filled capsules containing 15.0 mg oxycodone were subjected to dissolution tests equivalent to the dissolution test set forth in the USP 24 Monograph for oxycodone hydrochloride tablets, except that the testing medium was a USP grade buffer having a pH of 4.5, and a paddle speed of 75 rpm was used. Additionally, chromatographic separation of the oxycodone from the colorant in the gelatin capsule was necessary to overcome interference in the ultraviolet absorbance spectrum. The USP 24 Monograph for oxycodone hydrochloride tablets refers to the general dissolution test set forth in U.S. Pharmacopeia 24.

The following table summarizes the dissolution results obtained for the 15 mg oxycodone strength capsules based on 12 samples tested.

Dissolution Of Oxycodone Hydrochloride from Example 5

| Time (min) | Oxycodone Released (%) |
|---|---|
| 0 | 0 |
| 10 | 87 |
| 20 | 99 |
| 30 | 102 |

These dissolution results show that this dosage form provides immediate release of oxycodone hydrochloride.

Dissolution tests were also attempted at 50 rpm, but the paddle method did not provide enough agitation and coning occurred. At this lower paddle speed of 50 rpm, the overfill excipients from the capsule formed a cone at the bottom of the dissolution vessel and prevented the tablet from dissolving properly. The occurrence of coning justifies modification of the dissolution test parameters. To overcome the coning, the paddle speed was increased to 75 rpm, which is still within acceptable USP dissolution criteria.

### EXAMPLE 6

This example describes the preparation of a dosage form comprising tramadol hydrochloride (referred to in this example as “tramadol”) which was used in clinical evaluations. This example also describes the measurement of the dissolution of that dosage form. The following general procedure was used to prepare capsules containing different strengths of tramadol hydrochloride supplied as Ultram® (Johnson R W). Ultram® tablets are available in 50.0 mg (tramadol hydrochloride) strength tablets. Each Ultram® tablet is claimed to have a theoretical drug content of 50.0 mg tramadol hydrochloride, although the actual amount of each may vary within an acceptable range, for example by about 10%.

A 50.0 mg tramadol hydrochloride capsule was made by mixing microcrystalline cellulose (167.16 mg) with magnesium stearate (0.84 mg) to form a blend. The blend and one Ultram® tablet were loaded separately into the body component of a hard gelatin capsule shell. The capsule was then closed with the cap component of the shell. The filled capsule weighed approximately 491.3 mg.

The filled capsules were subjected to dissolution tests using Apparatus 2 (Paddle Stirring Element) in U.S. Pharmacopeia 24. Analysis of tramadol capsules was accomplished by performing dissolution on the filled capsules in 900 ml of 0.1 N hydrochloric acid. The media temperature was maintained at about 37.0° C. (+/−0.5° C.), and the paddle speed was 75 rpm. The following table summarizes the dissolution data obtained for the 50 mg tramadol hydrochloride strength capsules based on 6 samples having been tested.

Dissolution of Tramadol Hydrochloride from Example 6

| TIME (MIN) | TRAMADOL RELEASED (%) |
|---|---|
| 0 | 0 |
| 5 | 8 |
| 10 | 60 |
| 15 | 93 |
| 30 | 106 |

Once again, coning was observed for dissolution tests performed at 50 rpm which resulted in an unreliable test. To overcome the coning, the paddle speed was increased to 75 rpm, which is still within acceptable USP dissolution criteria.

These dissolution results show that this dosage form provides immediate release of tramadol hydrochloride and that the excipients employed do not significantly bind an opioid agonist in an aqueous environment.

### EXAMPLE 7

This example describes a general procedure used to make a pharmaceutical composition comprising an opioid antagonist on coated nonpareil beads or pellets, such as coated pellet 1 shown in FIG. 2. This example describes a dosage form consisting essentially of an opioid antagonist. A binder solution of at least a binder, an organic and/or aqueous solvent and an opioid antagonist is prepared. A suitable binder is hydroxypropyl methylcellulose (“HPMC”), plasticized with triacetin, e.g., Opadry® brand commercial coating from Colorcon, Inc. The binder solution is coated onto inert nonpareil pellets to form a coated pellet. The coating is conducted in a fluidized bed apparatus configured in a bottom-spray orientation with a Wurster column insert. A plasticizer may be added to the coating dispersion to increase the flexibility of the coating. The thickness (weight) of the coat on the nonpareil pellets can be varied as desired but generally falls in the range of about 2-50% wt. of the total pellet weight. The coated pellets weigh less than about 10 mg and have a diameter or length of about 0.1-5.0 mm. These coated pellets may comprise the following ingredients in the approximate amounts indicated.

Composition of Coated Pellets

| INGREDIENT | AMOUNT (% w/w) |
|---|---|
| Opioid antagonist (NTX) | 0.1 |
| Opadry ® Clear | 5.0 |
| Non-pareil sugar cores | 94.9 |

The nonpareil cores used in the coated pellet and bead formulations generally comprise a pharmaceutically inert material such as lactose, sucrose, and starch.

The coated pellets may be loaded into hard gelatin capsules or compressed into tablets.

### EXAMPLE 8

This example describes a general procedure used to make a pharmaceutical composition comprising a coated granulation similar to that shown in FIG. 6, except than the opioid antagonist is the only active pharmaceutical ingredient employed in this example. This example describes a dosage form consisting essentially of an opioid antagonist. A granulating-solution is made by mixing an opioid antagonist, one or more binders, and water in a vessel equipped with a high shear mixer. A suitable granulating solution comprises the following ingredients in the approximate amounts indicated.

Granulating Solution

| INGREDIENT | AMOUNT (% w/w) |
|---|---|
| Opioid antagonist (NTX) | 0.5 |
| HPMC E5 | 5.0 |
| PEG 8000 | 1.2 |
| Purified water | q.s. to 100 ml |

A first blend of inert pharmaceutical excipients (such as those in the table below) is then granulated with a granulating solution in a fluidized bed unit to form a granulated wet mass that is sized by passing it through a No. 12 sieve. The sized granules are subsequently dried, thereby forming the coated granulation, which is then passed through a No. 30 mesh screen.

Granulation Blend

| INGREDIENT | AMOUNT (% w/w) |
|---|---|
| HPMC | 4.5 |
| Lactose, hydrous | 73 |
| Dibasic calcium phosphate | 22.5 |

The granules are then mixed with magnesium stearate (0.5% wt.) and low-substituted hydroxypropylcelluose (“L-HPC”) (3.0% wt.) and blended for an additional 5 min. The blend is then compressed into tablets on a rotary tablet press.

### EXAMPLE 9

This example describes a general procedure used to make a soft gelatin capsule dosage form comprising a drug suspension. A suspension is made by mixing a non-aqueous vehicle such as miglyol, PEG, glycerin, propylene glycol, or vegetable oil with solids such as an opioid antagonist and optionally at least one other pharmaceutical excipient. The solids added to the non-aqueous vehicle are generally powdered or particulate and can include beads, pellets and granules, for example. A suitable suspension comprises the following ingredients in the approximate amounts indicated.

| INGREDIENT | AMOUNT (% w/w) |
|---|---|
| Opioid antagonist (NTX) | 0.05 |
| Talc | 10 |
| Miglyol 812 | q.s. to 100 |

### EXAMPLE 10

This example describes a general procedure used to make the pharmaceutical composition of FIG. 2, which comprises a mixture of two different coated nonpareil beads or pellets. This example describes a dosage form comprising an opioid antagonist and another active pharmaceutical ingredient (in this case, an opioid agonist). A combination capsule dosage form containing both naltrexone and morphine sulfate pentahydrate for concurrent release of both drugs was prepared. The excipients used in this example were found not to bind morphine or naltrexone significantly in an aqueous environment.

A first binder solution comprising at least a binder, an organic and/or aqueous solvent and an opioid antagonist is prepared. The first binder solution is coated onto inert nonpareil pellets to form a first coated pellet. A suitable binder is plasticizied HPMC, such as Opadry® Clear. The coating was conducted in a fluidized bed apparatus configured in a bottom-spray orientation with a Wurster column insert. The spray rate of the coating solutions was adjusted during processing to maintain the following approximate equilibrium conditions during processing; inlet temperature 70°-80° C., exhaust temperature 42°-47° C.

Composition of first Coated Pellets

| INGREDIENT | AMOUNT (% w/w) |
|---|---|
| Opioid antagonist (NTX) | 0.1 |
| Opadry ® Clear | 5.0 |
| Non-pareil sugar cores | 94.9 |

In a similar fashion, a second binder solution comprising at least a binder, an organic and/or aqueous solvent and an opioid agonist is prepared. The second binder solution is coated onto other inert nonpareil pellets to form a second coated pellet. A plasticizer may be added to the coating dispersion to increase the flexibility of the coating.

Aqueous coating solutions with the compositions listed above were used to manufacture capsules containing drug coated non-pareil beads that rapidly and concurrently release opioid agonist and opioid antagonist from the dosage form.

Composition of second Coated Pellets

| INGREDIENT | AMOUNT (% w/w/) |
|---|---|
| Opioid agonist (MS) | 7.5 |
| Opadry ® Clear | 7.5 |
| Non-pareil sugar cores | 85.0 |

Predetermined amounts of the first and second pellets are mixed thereby forming a mixture of the pellets. The thickness (weight) of the coat on the nonpareil pellets can be varied as desired but generally falls in the range of about 1-70% wt. of the total pellet weight. The coated pellets weigh less than about 10 mg and have a diameter or length of about 0.1-5.0 mm.

Capsules made according to this example contained the following ingredients in the approximate amounts indicated.

| | Mass Fraction | |
|---|---|---|
| INGREDIENT | (% w/w) | Quantity (mg) |
| Opioid Agonist Beads, consisting of: | | 400 |
| Opioid Agonist (morphine sulfate | 6.667 | 30.0 |
| pentahydrate) | | |
| Binder (e.g., plasticized HPMC) | 6.667 | 30.0 |
| Non-Pareil Sugar Cores | 75.556 | 340.0 |
| Opioid Antagonist Beads, consisting of: | | 50 |
| Opioid Antagonist (NTX) | 0.011 | 0.05 |
| Binder (e.g., plasticized HPMC) | 0.556 | 2.5 |
| Non-Pareil Sugar Cores | 10.544 | 47.45 |
| Total Net Content Weight | 100.000 | 450.0 |
| Hard Gelatin Capsule, Size 0 | | 1 capsule |

The nonpareil cores used in the coated pellet and bead formulations of the invention generally comprise a pharmaceutically inert material such as lactose, sucrose, and/or starch.

A mixture of coated pellets was loaded into hard gelatin capsules and subjected to dissolution tests using Apparatus 2 (Paddle Stirring Element) in U.S. Pharmacopeia 24, except that a variety of different agitation rates were used. The testing media was 500 ml of 0.1 N HCl, at a constant media temperature of 37° C. (+/−0.5° C.). The agitation rates of the paddles for the dissolution tests were 50, 75 or 100 rotations per minute. The tables below summarize the results obtained for the dissolution tests at the respective paddle speeds. The results reported at each time for each paddle speed are the mean values of six tests.

Dissolution profile at 50 rpm.

| Time (min) | MS Released (%) | NTX Released (%) |
|---|---|---|
| 0 | 0 | 0 |
| 10 | 55.3 | 60.4 |
| 20 | 72.1 | 72.0 |
| 30 | 82.1 | 98.7 |
| 45 | 89.5 | 95.1 |

Dissolution profile at 75 rpm.

| Time (min) | MS Released (%) | NTX Released (%) |
|---|---|---|
| 0 | 0 | 0 |
| 5 | 53.6 | 63.2 |
| 10 | 66.7 | 74.5 |
| 20 | 81.2 | 86.4 |
| 30 | 85.2 | 91.0 |
| 45 | 98.2 | 95.2 |
| 60 | 97.1 | 96.8 |

Dissolution profile at 100 rpm.

| Time (min) | MS Released (%) | NTX Released (%) |
|---|---|---|
| 0 | 0 | 0 |
| 5 | 68.6 | 69.6 |
| 10 | 84.3 | 82.6 |
| 20 | 97.2 | 91.6 |
| 30 | 101.2 | 95.4 |
| 45 | 102.7 | 96.1 |
| 60 | 103.1 | 98.1 |

These dissolution results show that this combination dosage form provides concurrent release of an opioid agonist and an opioid antagonist and that the excipients employed do not significantly bind an opioid agonist or an opioid agonist in an aqueous environment. These dissolution results show that in this combination dosage form, the dissolution profiles were substantially the same at each agitation rate, percentages of the opioid agonist and the opioid antagonist are substantially the same when tested at 75 rpm and 100 rpm at 5 minutes, 10 minutes, 20 minutes, 30 minutes, 45 minutes, and 60 minutes. The dosage form of this example also provides immediate release of both active pharmaceutical ingredients at greater than about 90% dissolution at 45 minutes.

### EXAMPLE 11

This example demonstrates a general procedure used to make the pharmaceutical composition of FIG. 3 comprising a single type of coated nonpareil pellet, wherein the coat comprises an opioid antagonist and another active pharmaceutical ingredient (in this case, an opioid agonist). A first binder solution comprising at least a binder, an organic and/or aqueous solvent, a plasticizer, an opioid agonist and an opioid antagonist is prepared. The first binder solution is coated onto inert nonpareil pellets to form coated pellets. The thickness (weight) of the coat on the nonpareil pellets can be varied as desired but generally falls in the range of about 2-80 % wt., normally between 10-50%, of the total pellet weight. The pellets weigh about less than 10 mg and have a diameter or length of about 0.2-2.0 mm. These coated pellets comprise the following ingredients in the approximate amounts indicated.

Coating Composition

| INGREDIENT | AMOUNT (% w/w) |
|---|---|
| Opioid antagonist (NTX) | 0.01 |
| Opioid agonist (MS) | 6 |
| HPC | 4.5 |
| PEG 8000 | 0.4 |
| Purified water | q.s. to 100 |

### EXAMPLE 12

This example demonstrates a combination tablet dosage form containing both naltrexone salt and morphine salt for concurrent release and immediate release of both active pharmaceutical ingredients. The combination tablet comprises an opioid antagonist and another active pharmaceutical ingredient (in this case, an opioid agonist). The excipients used in this example were found not to bind morphine or naltrexone significantly (e.g., to an extent that interferes with the therapeutic effect or concurrent release) in an aqueous environment. The tablet disintegrates within about 5 min after exposure to an aqueous buffer at 37° C. and provides rapid dissolution of the active pharmaceutical ingredients. Tablets made according to this example contained the following ingredients in the approximate amounts indicated.

Components of MS/NTX Concurrent Release Tablets

| | | Content |
|---|---|---|
| Component | Description | (mg/tablet) |
| Morphine Sulfate, Pentahydrate | API | 30.00 |
| Naltrexone HCl | API | 0.05 |
| Lactose Monohydrate | Filler | 92.51 |
| Dibasic calcium Phosphate, Dihydrate | Filler | 50.76 |
| Low-Substituted hydroxypropylcellulose | Disintegrant | 16.87 |
| Hydroxypropyl methylcellulose 2910 | Binder | 2.81 |
| Talc | Glidant | 6.00 |
| Magnesium stearate (comperdial | Lubricant | 1.00 |
| designations are not used elsewhere) | | |

The tablets were made according to the following general procedure. A high shear, wet granulation method was employed to prepare the bulk powders for tableting.

Granulating solution

| | AMOUNT IN SOLUTION |
|---|---|
| INGREDIENT | (% w/w) |
| Opioid antagonist (NTX) | 0.265 |
| Hydroxypropyl Methylcellulose 2910 | 5.0 |
| Purified water | q.s. to 100 ml |

A dry material blend of an opioid agonist and the following pharmaceutical excipients was prepared in a high shear granulator.

Granulation Blend

| | AMOUNT IN BLEND |
|---|---|
| INGREDIENT | (% w/w) |
| Opioid agonist (MS) | 16.95 |
| Hydroxypropyl Methylcellulose 2910 | 1.06 |
| Low Substituted Hydroxypropyl | 1.06 |
| Cellulose (L-HPC) | |
| Lactose Monohydrate, Spray Dried | 52.26 |
| Dibasic calcium phosphate | 28.68 |

While mixing, the binder solution was added slowly to the dry material blend to prepare the granulation. The wet granulation was sized by passing it through a 20 mesh screen. The sized wet granulation was dried in a fluid-bed apparatus until the dried mass had a moisture content of less than about five percent. The dried granulation was passed through a 20 mesh screen. The following non-granulated excipients were blended into the dried granulation using a double cone blender.

Non-granulated Excipients

| | AMOUNT IN FINAL |
|---|---|
| INGREDIENT | EXCIPIENTS (% w/w) |
| Talc | 27.27 |
| L-HPC | 68.18 |
| Magnesium Stearate | 4.55 |

The final powder blend was then compressed on a rotary tablet press using 7 mm round, biconcave tooling. The compressed tablet compacts were collected, and the aqueous based coatings applied in a tablet-coating unit.

The tablets were coated with a clear seal coat of plasticized HPMC (Opadry®) based aqueous coating solution and a colored topcoat of plasticized HPMC (Opadry®) based aqueous coating dispersion.

The tablets were determined to disintegrate in less than 3 minutes in deionized water at 37° C. (+/−2° C.) using the disintegration test in U.S. Pharmacopeia 24 (2000) Physical Tests <701> (which is incorporated herein by reference).

The dissolution of the MS and NTX from the uncoated and coated tablet cores was evaluated using Apparatus 2 (Paddle Stirring Element) in U.S. Pharmacopeia 24, with 500 mL of 0.1N HCl as the dissolution media. Two tablets were placed in 500 mL of dissolution media at 37° C. (+/−0.5° C.). The paddle agitation rate was 50 rpm, and the percentages of dissolved MS and NTX were determined from samples of the testing medium.

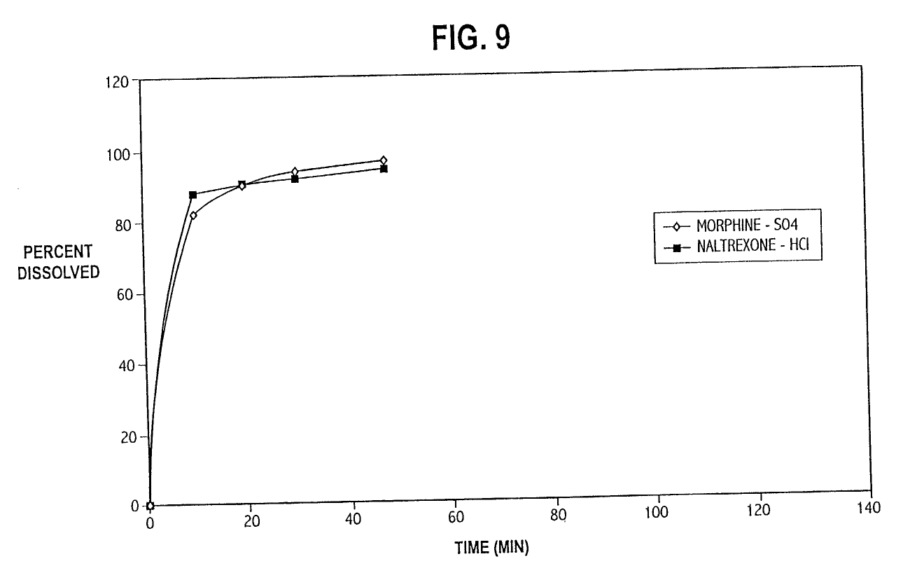

The tables below summarize the results of the dissolution tests on the uncoated tablet cores and the coated tablet cores prepared as described above. FIG. 9 shows the dissolution profile of the coated tablet cores.

Dissolution of MS and NTX from Uncoated,
Concurrent Release Tablets

| Time (min) | % MS Released | % NTX Released |
|---|---|---|
| 0 | 0 | 0 |
| 10 | 82.4 | 97.7 |
| 20 | 95.1 | 95.6 |
| 30 | 96.4 | 119.8 |
| 45 | 96.2 | 103.3 |

Dissolution of MS and NTX from Coated,
Concurrent Release Tablets

| Time (min) | % MS Released | % NTX Released |
|---|---|---|
| 0 | 0 | 0 |
| 10 | 82.9 | 89.3 |
| 20 | 90.0 | 90.3 |
| 30 | 94.1 | 92.9 |
| 45 | 96.3 | 95.5 |

Accordingly, this embodiment of the invention provides an immediate release solid oral tablet comprising an opioid antagonist and an opioid agonist. The active pharmaceutical ingredients are released concurrently. Furthermore, the dissolution percentages of the opioid agonist and the opioid antagonists in this dosage form are substantially the same at each time (except at 30 min. for the uncoated tablets). These dissolution results show that this combination dosage form provides concurrent release of an opioid agonist and an opioid antagonist and that the excipients employed do not significantly bind an opioid agonist in an aqueous environment.

### EXAMPLE 13

This example demonstrates a general procedure used to make the pharmaceutical composition of FIG. 4 comprising a mixture of two different types of granulations. One granulation comprises an opioid antagonist and the other granulation comprises another active pharmaceutical ingredient (in this case, an opioid agonist).

A first granulation is made by blending an opioid antagonist and a first blend of pharmaceutical excipients with a granulating solution to form a wet mass that is passed through a sieve to form wet granules that are subsequently dried. A suitable first granulation comprises the following ingredients in the approximate amounts indicated.

First Granulation

| INGREDIENT | AMOUNT (% w/w) |
|---|---|
| Opioid antagonist (NTX) | 0.5 |
| HPMC E5 | 5 |
| Lactose, hydrous | 54.5 |
| Encompress ™ | 40 |

Likewise, a second granulation is made by blending an opioid agonist and a second blend of pharmaceutical excipients with a granulating solution to form a wet mass that is passed through a sieve to form wet granules that are subsequently dried. A suitable second granulation comprises the following ingredients in the approximate amounts indicated.

Second Granulation

| INGREDIENT | AMOUNT (% w/w) |
|---|---|
| Opioid agonist (MS) | 10 |
| HPMC E5 | 4.5 |
| L-HPC | 2 |
| Lactose, hydrous | q.s. to 100 |

The first and second granulations are subsequently blended to form the mixture. The granulations pass through a No. 20 mesh screen. Granulations of both active pharmaceutical ingredients are passed through a No. 20 mesh screen and blended with a disintegrant (e.g., L-HPC, 3% wt.) and a lubricant (e.g., magnesium stearate, 0.5% wt.) for 10 min in a twin-shell blender prior to compression on a rotary tablet press.

### EXAMPLE 14

This example demonstrates a general procedure used to make the pharmaceutical composition of FIG. 5 comprising a combination granulation. The combination granulation comprises an opioid antagonist and another active pharmaceutical ingredient (in this case, an opioid agonist).

A first granulation is made by blending an opioid antagonist and a first blend of pharmaceutical excipients with a granulating solution to form a wet mass that is passed through a No. 12 sieve to form wet granules that are subsequently dried. The final granulation passes through a No. 20 mesh screen. A suitable first granulation comprises the following ingredients in the approximate amounts indicated.

First Granulation

| INGREDIENT | AMOUNT (% w/w) |
|---|---|
| Opioid antagonist (NTX) | 0.5 |
| HPMC E5 | 3.75 |
| Sucrose | 5 |
| Lactose | 80 |
| Dicalcium phosphate | 10 |

A second granulation is made by blending an opioid agonist, a second blend of pharmaceutical excipients and the first granulation with a granulating solution to form a wet mass that is passed through a sieve to form wet granules that are subsequently dried. A suitable second granulation comprises the following ingredients in the approximate amounts indicated.

Second Granulation

| INGREDIENT | AMOUNT (% w/w) |
|---|---|
| First Granulation | 20 |
| Opioid Agonist (Hydrocodone bitartrate) | 4.0 |
| HPMC E5 | 4.5 |
| L-HPC | 1.5 |
| Lactose, hydrous | q.s. to 70 |

### EXAMPLE 15

This example demonstrates a general procedure used to make the pharmaceutical composition of FIG. 6 comprising a coated granulation which comprises an opioid antagonist and another active pharmaceutical ingredient (in this case, an opioid agonist). A granulating solution is made by blending an opioid agonist, an opioid antagonist, a binder and a solvent in a vessel equipped with a high shear mixer. A suitable granulating solution comprises the following ingredients in the approximate amounts indicated.

Granulating solution

| INGREDIENT | AMOUNT (% w/w) |
|---|---|
| Opioid antagonist (NTX) | 0.1 |
| Opioid agonist (hydrocodone bitartrate) | 10 |
| HPMC E5 | 5.5 |
| PEG 8000 | 1.0 |
| Purified water | q.s. to 100 |

A first blend of inert pharmaceutical excipients is then granulated with the granulating solution in a fluidized bed unit to form a wet mass that is passed through a No. 12 sieve to form wet granules which are subsequently dried, thereby forming the coated granulation.

The final granulation is then mixed with one or more pharmaceutical excipients to form a pharmaceutical mixture comprising the following ingredients in the approximate amounts indicated. The final granulation passes through a No. 20 mesh screen.

Granulation

| INGREDIENT | AMOUNT (% w/w) |
|---|---|
| Weight of solids from granulating | 15 |
| solution | |
| Lactose | 39 |
| Encompress | 41.5 |
| L-HPC | 4 |
| Magnesium stearate | 0.5 |

### EXAMPLE 16

This example demonstrates a general procedure similar to the one used to make the pharmaceutical composition of Example 15 except that the opioid agonist is not included in the granulating solution and is instead included with the pharmaceutical excipients being granulated.

A granulating-solution is made by mixing an opioid antagonist, a binder and a solvent in a vessel equipped with a high shear mixer. A suitable granulating-solution comprises the following ingredients in the approximate amounts indicated.

Granulating solution

| INGREDIENT | AMOUNT (% w/w) |
|---|---|
| Opioid antagonist (NTX) | 0.5 |
| HPMC E5 | 5.0 |
| PEG 800 | 1.2 |
| Purified water | q.s. to 100 ml |

A first blend of inert pharmaceutical excipients is then granulated with the granulating solution in a fluidized bed unit to form a wet mass that is passed through a No. 12 sieve to form wet granules which are subsequently dried, thereby forming the coated granulation, and passed through a No. 30 mesh screen. A suitable granulation comprises the following ingredients in the approximate amounts indicated.

Granulation

| INGREDIENT | AMOUNT (% w/w) |
|---|---|
| Opioid agonist (oxycodone bitartrate) | 5.0 |
| HPMC | 4.5 |
| Lactose, hydrous | 68 |
| Dibasic calcium phosphate | 22.5 |

The coated granulation is mixed with magnesium stearate (0.5% wt.) and L-HPC (3.0% wt.) and blended for an additional 5 min. The blend is then compressed into tablets on a rotary tablet press.

### EXAMPLE 17

This example demonstrates a general procedure used to make a pharmaceutical composition comprising spray-dried granules. A solution of an opioid antagonist, another active pharmaceutical ingredient, such as an opioid agonist, and at least one pharmaceutical excipient is spray-dried in a BUCHI™ spray-dryer at a rate of about 10 ml/min while using an inlet temperature of 90° C. and an outlet temperature of 50° C. The spray-dried granules are collected and have a particle size of less than about 20 mesh. Suitable spray-dried granules comprise the following ingredients in the approximate amounts indicated.

| INGREDIENT | AMOUNT (% w/w) |
|---|---|
| Opioid agonist (oxycodone | 10 |
| hydrochloride) | |
| Opioid antagonist (NTX) | 0.2 |
| HPMC E5 | 3 |
| Sucrose: lactose (1:1) | q.s. to 86.8 |

While specific parameters for operation of the spray-dryer are detailed above, operation of the spray-dryer can be conducted as needed and the parameters for operation of the apparatus can be varied as needed to prepare a product according to this example.

The granules are dried and blended with magnesium stearate (0.5% wt.) and L-HPC (3.0% wt.) and blended for an additional 4-6 min. The blend is then compressed into tablets on a rotary tablet press or is filled into hard gelatin capsules.

### EXAMPLE 18

This example demonstrates a general procedure used to make a soft gelatin capsule dosage form containing a drug suspension. A suspension is made by mixing a non-aqueous vehicle such as miglyol, PEG, glycerin, propylene glycol, or vegetable oil with an opioid antagonist, another active pharmaceutical ingredient such as an opioid agonist, and optionally at least one other pharmaceutical excipient. The solids added to the non-aqueous vehicle are generally powdered or particulate and can include beads, pellets and granules, for example. A suitable suspension comprises the following ingredients in the approximate amounts indicated.

| INGREDIENT | AMOUNT (% w/w) |
|---|---|
| Opioid agonist (MS) | 20 |
| Opioid antagonist (NTX) | 0.05 |
| Talc | 10 |
| Miglyol 812 | q.s to 100 |

Although the invention herein has been described with reference to particular embodiments, it is to be understood that these embodiments are merely illustrative of various aspects of the invention. Thus, it is to be understood that numerous modifications may be made in the illustrative embodiments and other arrangements may be devised without departing from the spirit and scope of the invention.

What is claimed is:

1. A dosage form comprising an opioid antagonist in an amount from about 0.0001 mg to about 1 mg or less than 1 mg.

2. The dosage form of claim 1 wherein the amount of antagonist is about 1 mg.

3. The dosage form of claim 1 wherein the amount of antagonist is less than 1 mg.

4. The dosage form of claim 1 wherein the amount of antagonist is less than about 0.5 mg.

5. The dosage form according to claims 1 or 4 wherein the amount of antagonist is about 0.1 mg.

6. The dosage form according to claims 1 or 4 wherein the amount of antagonist is less than 0.1 mg.

7. The dosage form according to claims 1 or 4 wherein the amount of antagonist is more than 0.1 mg.

8. The dosage form according to claims 1 or 4 wherein the amount of antagonist is about 0.01 mg.

9. The dosage form according to claims 1 or 4 wherein the amount of antagonist is less than 0.01 mg.

10. The dosage form according to claims 1 or 4 wherein the amount of antagonist is more than 0.01 mg.

11. The dosage form according to claims 1 or 4 wherein the amount of antagonist is about 0.001 mg.

12. The dosage form according to claims 1 or 4 wherein the amount of antagonist is less than 0.001 mg.

13. The dosage form according to claims 1 or 4 wherein the amount of antagonist is more than 0.001 mg.

14. The dosage form according to claims 1 or 4 wherein the amount of antagonist is about 0.0001 mg.

15. The dosage form according to claims 1 or 4 wherein the amount of antagonist is more than 0.0001 mg.

16. The dosage form according to claims 1 or 4 wherein the antagonist is present as the pharmaceutically acceptable salt.

17. The dosage form according to claims 1 or 4 wherein the antagonist is naltrexone, nalmefene or naloxone.

18. The dosage form according to claims 1 or 4 further comprising an opioid agonist.

19. The dosage form of claim 18 wherein the agonist is in an analgesic or subanalgesic amount.

20. The dosage form of claim 18 wherein the agonist is morphine, hydrocodone, oxycodone, codeine, fentanyl, alfentanil, hydromorphone, meperidine, methadone, oxymorphone, propoxyphene or tramadol.

21. The dosage form of claim 18 wherein the agonist is morphine, hydrocodone, oxycodone, or tramadol.

22. The dosage form according to claims 1 or 4 wherein the antagonist is formulated as a capsule, tablet or pill.

23. A pharmaceutical composition comprising an opioid antagonist in an amount from about 0.0001 mg to about 1 mg or less than 1 mg and a pharmaceutically acceptable carrier.

24. The pharmaceutical composition of claim 23 wherein the amount of antagonist is about 1 mg.

25. The pharmaceutical composition of claim 23 wherein the amount of antagonist is less than 1 mg.

26. The pharmaceutical composition of claim 23 wherein the amount of antagonist is about 0.1 mg.

27. The pharmaceutical composition of claim 23 wherein the amount of antagonist is less than 0.1 mg.

28. The pharmaceutical composition of claim 23 wherein the amount of antagonist is more than 0.1 mg.

29. The pharmaceutical composition of claim 23 wherein the amount of antagonist is about 0.01 mg.

30. The pharmaceutical composition of claim 23 wherein the amount of antagonist is less than 0.01 mg.

31. The pharmaceutical composition of claim 23 wherein the amount of antagonist is more than 0.01 mg.

32. The pharmaceutical composition of claim 23 wherein the amount of antagonist is about 0.001 mg.

33. The pharmaceutical composition of claim 23 wherein the amount of antagonist is less than 0.001 mg.

34. The pharmaceutical composition of claim 23 wherein the amount of antagonist is more than 0.001 mg.

35. The pharmaceutical composition of claim 23 wherein the amount of antagonist is about 0.0001 mg.

36. The pharmaceutical composition of claim 23 wherein the amount of antagonist is more than 0.0001 mg.

37. The pharmaceutical composition of claim 23 wherein the antagonist is present as the pharmaceutically acceptable salt.

38. The pharmaceutical composition of claim 23 wherein the antagonist is naltrexone, nalmefene or naloxone.

39. The pharmaceutical composition of claim 23 further comprising an opioid agonist.

40. The pharmaceutical composition of claim 39 wherein the agonist is in an analgesic or subanalgesic amount.

41. The dosage form of claim 39 wherein the agonist is morphine, hydrocodone, oxycodone, codeine, fentanyl, alfentanil, hydromorphone, meperidine, methadone, oxymorphone, propoxyphene or tramadol.

42. The pharmaceutical composition of claim 39 wherein the agonist is morphine, hydrocodone, oxycodone or tramadol.

43. The pharmaceutical composition according to claims 23 or 39 wherein the antagonist is formulated as a capsule, tablet or pill.

44. A method of administering a therapeutic dose of an opioid antagonist to a human subject comprising administering the dosage form according to claims 1 or 4.

45. A method of administering a therapeutic dose of an opioid antagonist to a human subject comprising administering the dosage form of claims 18.

46. A pharmaceutical kit comprising a dosage form according to claims 1 or 4.

47. A pharmaceutical kit comprising a dosage form of claim 18 and a container.

48. A solid oral dosage form consisting essentially of an opioid antagonist in an amount from about 0.0001 mg to less than 0.5 mg.

49. The solid oral dosage form of claim 48 wherein the amount of antagonist is less than about 0.4 mg.

50. The solid oral dosage form of claim 48 wherein the amount of antagonist is less than about 0.3 mg.

51. The solid oral dosage form of claim 48 wherein the amount of antagonist is less than about 0.2 mg.

52. The solid oral dosage form of claim 48 wherein the amount of antagonist is about 0.1 mg.

53. The solid oral dosage form of claim 48 wherein the amount of antagonist is less than 0.1 mg.

54. The solid oral dosage form of claim 48 wherein the amount of antagonist is more than 0.1 mg.

55. The solid oral dosage form of claim 48 wherein the amount of antagonist is about 0.01 mg.

56. The solid oral dosage form of claim 48 wherein the amount of antagonist is less than 0.01 mg.

57. The solid oral dosage form of claim 48 wherein the amount of antagonist is more than 0.01 mg.

58. The solid oral dosage form of claim 48 wherein the amount of antagonist is about 0.001 mg.

59. The solid oral dosage form of claim 48 wherein the amount of antagonist is less than 0.001 mg.

60. The solid oral dosage form of claim 48 wherein the amount of antagonist is more than 0.001 mg.

61. The solid oral dosage form of claim 48 wherein the amount of antagonist is about 0.0001 mg.

62. The solid oral dosage form of claim 48 wherein the amount of antagonist is more than 0.0001 mg.

63. The solid oral dosage form of claim 48 wherein the antagonist is naltrexone or nalmefene.

64. A solid oral dosage form consisting essentially of naltrexone in an amount from about 0.0001 mg to less than about 0.5 mg.

65. The solid oral dosage form of claim 64 wherein the amount of naltrexone is less than an effective antagonistic amount.

66. A solid oral dosage form consisting essentially of nalmefene in an amount from about 0.0001 mg to less than about 0.5 mg.

67. The solid oral dosage form of claim 66 wherein the amount of nalmefene is less than an effective antagonistic amount.

68. A solid oral dosage form comprising an opioid antagonist in an amount from about 0.0001 mg to less than about 0.5 mg and an opioid agonist in an amount from about 0.1 mg to about 300 mg.

69. The solid oral dosage form of claim 68 wherein the antagonist is present in less than an effective antagonistic amount.

70. The solid oral dosage form of claim 68 wherein the amount of antagonist is about 0.1 mg.

71. The solid oral dosage form of claim 68 wherein the amount of antagonist is less than 0.1 mg.

72. The solid oral dosage form of claim 68 wherein the amount of antagonist is more than 0.1 mg.

73. The solid oral dosage form of claim 68 wherein the amount of antagonist is about 0.01 mg.

74. The solid oral dosage form of claim 68 wherein the amount of antagonist is less than 0.01 mg.

75. The solid oral dosage form of claim 68 wherein the amount of antagonist is more than 0.01 mg.

76. The solid oral dosage form of claim 68 wherein the amount of antagonist is about 0.001 mg.

77. The solid oral dosage form of claim 68 wherein the amount of antagonist is less than 0.001 mg.

78. The solid oral dosage form of claim 68 wherein the amount of antagonist is more than 0.001 mg.

79. The solid oral dosage form of claim 68 wherein the amount of antagonist is about 0.0001 mg.

80. The solid oral dosage form of claim 68 wherein the amount of antagonist is more than 0.0001 mg.

81. The solid oral dosage form of claim 68 wherein the antagonist is present as a pharmaceutically acceptable salt.

82. The solid oral dosage form of claim 68 wherein the antagonist is naltrexone or nalmefene.

83. The solid oral dosage form of claim 68 comprising naltrexone as the antagonist.

84. The solid oral dosage form of claim 68 comprising nalmefene as the antagonist.

85. The solid oral dosage form of claim 68 wherein the agonist is present in a subanalgesic amount.

86. The dosage form of claim 68 wherein the agonist is morphine, hydrocodone, oxycodone, codeine, fentanyl, alfentanil, hydromorphone, meperidine, methadone, oxymorphone, propoxyphene or tramadol.

87. The solid oral dosage form of claim 68 wherein the agonist is morphine, hydrocodone, oxycodone or tramadol.

88. The solid oral dosage form of claim 68, wherein the antagonist is naltrexone and the agonist is morphine.

89. The solid oral dosage form of claim 68, wherein the antagonist is naltrexone and the agonist is oxycodone.

90. The solid oral dosage form of claim 68 wherein the antagonist is naltrexone and the agonist is hydrocodone.

91. The solid oral dosage form of claim 68, wherein the antagonist is naltrexone and the agonist is tramadol.

92. The solid oral dosage form of claim 68, wherein the antagonist is nalmefene and the agonist is morphine.

93. The solid oral dosage form of claim 68, wherein the antagonist is nalmefene and the agonist is oxycodone.

94. The solid oral dosage form of claim 68, wherein the antagonist is nalmefene and the agonist is tramadol.

95. The solid oral dosage from of claim 68 wherein the antagonist is nalmefene and the agonist is hydrocone.

96. The solid oral dosage form of claim 68, wherein the antagonist is formulated as a capsule, tablet or pill.

97. The solid oral dosage form of claim 68 additionally comprising another active pharmaceutical ingredient selected from the group of acetaminophen, steroidal anti-inflammatory drugs, non-steroidal anti-inflammatory drugs, COX-1 inhibitors and COX-2 inhibitors.

98. A method of administering a therapeutic dose of an opioid antagonist to a human subject consisting essentially of administering the solid oral dosage form according to claim 22.

99. A method of administering a therapeutic dose of an opioid antagonist to a human subject consisting essentially of administering the solid oral dosage form according to claim 23.

100. A method of administering a therapeutic dose of an opioid antagonist to a human subject consisting essentially of administering the solid oral dosage form according to claim 42.

101. A method of administering a therapeutic dose of an opioid antagonist to a human subject consisting essentially of administering the solid oral dosage form according to claim 43.

102. A method of administering a therapeutic dose of an opioid antagonist to a human subject consisting essentially of administering the solid oral dosage form according to claim 46.

103. A method of administering a therapeutic dose of an opioid antagonist to a human subject consisting essentially of administering the solid oral dosage form according to claim 62.

104. A method of administering a therapeutic dose of an opioid antagonist to a human subject consisting essentially of administering the solid oral dosage form according to claim 68.

105. A pharmaceutical kit comprising a dosage form according to claims 1, 4 or 46.

106. The kit of claim 105, further comprising a dosage form of an opioid agonist.

107. The kit of claim 106, wherein the agonist dosage form and the antagonist dosage form are separate dosage forms.

108. The solid oral dosage form according to claims 1 or 4, provided in an immediate release formulation.

109. The solid oral dosage form according to claim 22, provided in an immediate release formulation.

110. The solid oral dosage form according to claim 43, provided in an immediate release formulation.

111. The solid oral dosage form according to claim 48, provided in an immediate release formulation.

112. The solid oral dosage form according to claim 64, provided in an immediate release formulation.

113. The solid oral dosage form according to claim 68, provided in an immediate release formulation.

114. An immediate release solid oral dosage form comprising an opioid antagonist present in an amount from about 0.0001 to less than about 0.5 mg; at least one pharmaceutical excipient; wherein greater than 90% of the opioid antagonist is released in less than about 45 minutes after exposure to an aqueous environment.

115. The immediate release solid oral dosage form of claim 114, wherein greater than 90% of the opioid antagonist is released in less than about 30 minutes after exposure to an aqueous environment.

116. The immediate release solid oral dosage form of claim 114, wherein greater than 90% of the opioid antagonist is released in less than about 20 minutes after exposure to an aqueous environment.

117. The solid oral dosage form of claim 114 wherein the dosage form comprises a pharmaceutical composition comprising a coated solid substrate, the solid substrate comprising: an inert substrate; a coat at least partially surrounding the inert substrate; and
the coat comprises the opioid antagonist and at least one pharmaceutical excipient, and optionally a binder and a plasticizer.

118. The solid oral dosage form of claim 117, wherein the coat comprises about 1-70% wt. of the coated solid substrate.

119. The dosage form of claim 118, wherein the pharmaceutical composition further comprises at least one pharmaceutical excipient in admixture with the coated solid substrate.

120. The solid oral dosage form of claim 118, wherein the dosage form is a capsule or tablet.

121. The solid oral dosage form of claim 114 wherein the solid dosage form comprises a pharmaceutical composition comprising a coated granulation, the coated granulation comprising: granules comprising a mixture of two or more pharmaceutical excipients; and a coat at least partially surrounding each of the granules; and the coat comprises the pharmaceutical excipient and the opioid antagonist.

122. The solid oral dosage form of claim 121 wherein the pharmaceutical composition further comprises at least one pharmaceutical excipient in admixture with the coated granulation.

123. The solid oral dosage form of claim 122 wherein the coated granulation comprises about 10-85% wt. of the solid pharmaceutical composition.

124. The solid oral dosage form of claim 123 wherein the dosage form is a capsule or tablet.

125. The solid oral dosage form of claim 114 wherein the solid dosage form comprises a spray-dried pharmaceutical composition comprising a mixture of the at least one pharmaceutical excipient and the opioid antagonist.

126. The dosage form of claim 125 wherein the pharmaceutical composition further comprises at least one pharmaceutical excipient in admixture with the spray-dried pharmaceutical composition.

127. The solid oral dosage form of claim 126 wherein the spray-dried pharmaceutical composition comprises about 5-99% wt. of the dosage form.

128. The solid oral dosage form of claim 127, wherein the dosage form is a capsule or tablet.

129. The solid oral dosage form of claim 128 wherein the solid dosage form comprises a suspension-filled soft gelatin capsule comprising: a soft gelatin capsule; and a suspension within the capsule comprising a non-aqueous vehicle in liquid form and an opioid antagonist in solid form.

130. The dosage form of claim 128 wherein the suspension further comprises at least one pharmaceutical excipient.

131. An immediate release solid oral dosage form comprising: an opioid antagonist present in an amount from about 0.0001 mg to less than 0.5 mg; another active pharmaceutical ingredient; at least one pharmaceutical excipient; wherein the opioid antagonist and the other active ingredient are released in less than about 1.5 hours after exposure to an aqueous environment.

132. The solid oral dosage form of claim 131 wherein the other active pharmaceutical ingredient is an opioid agonist present in an amount from about 0.1 mg to about 300 mg.

133. A concurrent release combination solid oral dosage form comprising: an opioid antagonist present in an amount from about 0.0001 mg to less than about 0.5 mg; another active pharmaceutical ingredient; at least one pharmaceutical excipient; wherein the opioid antagonist and the other active pharmaceutical ingredient are released concurrently.

134. The solid oral dosage form of claim 133, wherein greater than 90% of each of the opioid antagonist and the other active pharmaceutical ingredient are released in less than about 45 minutes after exposure to an aqueous environment.

135. The solid oral dosage form of claim 133 wherein the other active ingredient is an opioid agonist present in an amount from about 0.1 mg to about 300 mg.

136. The solid oral dosage form according to claims 131 or 133 wherein the dosage form is a capsule or tablet.

137. The solid oral dosage form according to claims 131 or 133 wherein greater than 90% of the opioid antagonist is released in less than about 45 minutes after exposure to an aqueous environment.

138. The solid oral dosage form according to claims 131 or 133 wherein greater than 90% of the other active pharmaceutical ingredient is released in less than about 45 minutes after exposure to an aqueous environment.

139. The solid oral dosage form of claim 133 wherein a majority of the release of the opioid antagonist overlaps a majority of the release of the other active pharmaceutical ingredient.

140. The solid oral dosage form of claim 133 wherein about 90% of the opioid antagonist is released within a time period in which about 90% of the other active pharmaceutical ingredient is released.

141. The solid oral dosage form of claim 133, wherein the dosage form provides an antagonist dissolution profile and an other active pharmaceutical ingredient dissolution profile, and the dissolution profiles are substantially the same.

142. A combination solid oral dosage form comprising: an opioid agonist; an opioid antagonist; at least one pharmaceutical excipient; wherein the dosage form provides an agonist dissolution percentage and an antagonist dissolution percentage, as measured by the USP Paddle Method at 75 rpm in 500 ml of 0.1 HCl media at about 37° C., and the agonist dissolution percentage and the antagonist dissolution percentage are substantially the same at any time from 5 minutes to 30 minutes.

143. The solid oral dosage form of claim 142 wherein the agonist dissolution percentage and the antagonist dissolution percentage are substantially the same at each of 5 minutes, 10 minutes, 15 minutes, 20 minutes and 30 minutes.

144. The solid oral dosage form of claim 142 wherein the agonist dissolution percentage and the antagonist dissolution percentage are the means of at least six measurements.

145. The solid oral dosage form of claim 144 wherein the difference between the agonist dissolution percentage and the antagonist dissolution percentage is about 10% or less.

146. A combination solid oral dosage form comprising: an opioid antagonist present in an amount from about 0.0001 mg to less than about 0.5 mg; an opioid agonist present in an amount from about 0.1 mg to about 300 mg; and at least one pharmaceutical excipient; wherein the dosage form comprises a pharmaceutical composition comprising a coated solid substrate, the coated solid substrate comprising: a solid substrate comprising the opioid agonist and at least one pharmaceutical excipient; and a coat at least partially surrounding the solid substrate, and the coat comprises the opioid antagonist and at least one pharmaceutical excipient.

147. The solid oral dosage form according to claim 146, wherein the solid substrate is a pellet, bead or granule.

148. A combination solid oral dosage form comprising: a opioid antagonist present in an amount from about 0.0001 mg to less than about 0.5 mg; an opioid agonist present in an amount from about 0.1 mg to about 300 mg; and at least one pharmaceutical excipient; wherein the dosage form comprises a pharmaceutical composition comprising a coated solid substrate, the coated solid substrate comprising: an inert nonpareil bead or pellet; a coat at least partially surrounding the bead or pellet, and the coat comprises the opioid antagonist, the opioid agonist, a binder, a plasticizer and at least one pharmaceutical excipient.

149. A combination solid oral dosage form comprising: an opioid antagonist present in an amount from about 0.0001 mg to less than about 0.5 mg; an opioid agonist present in an amount from about 0.1 mg to about 300 mg; and at least one pharmaceutical excipient; wherein the solid dosage form comprises a pharmaceutical composition comprising first and second coated solid substrates, and: the first coated solid substrate comprises a nonpareil pellet or bead and a first coat at least partially surrounding the pellet or bead and the first coat comprises a binder and an opioid antagonist; and the second coated solid substrate comprises a nonpareil pellet or bead and a second coat at least partially surrounding the pellet or bead, and the second coat comprises a binder and an opioid agonist.

150. The solid oral dosage form of claim 149, wherein the first coated solid substrate comprises about 0.5-80% wt. of the solid pharmaceutical composition; and the second coated solid substrate comprises about 20-95% wt. of the solid pharmaceutical composition.

151. The solid oral dosage form of claim 150, wherein the pharmaceutical composition further comprises at least one pharmaceutical excipient in admixture with the first and second coated solid substrates.

152. A combination solid oral dosage form comprising: an opioid antagonist present in an amount from about 0.0001 mg to less than about 0.5 mg; an opioid agonist present in an amount from about 0.1 mg to about 300 mg; and at least one pharmaceutical excipient; wherein the solid dosage form comprises a pharmaceutical composition comprising first and second granulations; and the first granulation comprises a binder and the opioid antagonist; and the second granulation comprises a binder and the opioid agonist.

153. The solid oral dosage form of claim 152, wherein the first granulation comprises about 0.5-50% wt. of the solid pharmaceutical composition; and the second granulation comprises about 20-95% wt. of the solid pharmaceutical composition.

154. The solid oral dosage form of claim 153, wherein the pharmaceutical composition further comprises at least one pharmaceutical excipient in admixture with the first and second granulations.

155. The solid oral dosage form of claim 154, wherein the dosage form is a capsule or tablet.

156. A combination solid oral dosage form comprising: an opioid antagonist present in an amount from about 0.0001 mg to less than about 0.5 mg; an opioid agonist present in an amount from about 0.1 mg to about 300 mg; and at least one pharmaceutical excipient; wherein the solid dosage form comprises a pharmaceutical composition comprising a coated granulation comprising a mixture of two or more pharmaceutical excipients; and a coat at least partially surrounding the mixture, and the coat comprises a binder, an opioid agonist and an opioid antagonist.

157. The solid oral dosage form of claim 156, wherein the pharmaceutical composition further comprises at least one pharmaceutical excipient in admixture with the coated granulation.

158. The solid oral dosage form of claim 157, wherein the coated granulation comprises about 10-85% wt. of the solid pharmaceutical composition.

159. The solid oral dosage form of claim 158, wherein the dosage form is a capsule or tablet.

160. A combination solid oral dosage form comprising: an opioid antagonist present in an amount from about 0.0001 mg to less than about 0.5 mg; an opioid agonist present in an amount from about 0.1 mg to about 300 mg; and at least one pharmaceutical excipient; wherein the solid dosage form comprises a pharmaceutical composition comprising a coated granulation comprising a mixture of two or more pharmaceutical excipients and the opioid agonist; and a coat at least partially surrounding the mixture, and the coat comprises a binder and the opioid antagonist.

161. The solid oral dosage form of claim 160, wherein the pharmaceutical composition further comprises at least one pharmaceutical excipient in admixture with the coated granulation.

162. The solid oral dosage form of claim 161, wherein the coated granulation comprises about 10-75% wt. of the solid pharmaceutical composition.

163. The solid oral dosage form of claim 162, wherein the dosage form is a capsule or tablet.

164. A combination solid oral dosage form comprising: an opioid antagonist present in an amount from about 0.0001 mg to less than about; an opioid agonist present in an amount from about 0.1 mg to about 300 mg; and at least one pharmaceutical excipient; wherein the solid dosage form comprises a spray-dried pharmaceutical composition comprising a mixture of the at least one pharmaceutical excipient, the opioid agonist and the opioid antagonist.

165. The solid oral dosage form of claim 164, wherein the pharmaceutical composition further comprises at least one pharmaceutical excipient in admixture with the spray-dried pharmaceutical composition.

166. The solid oral dosage form of claim 165, wherein the spray-dried pharmaceutical composition comprises about 5-99% wt. of the dosage form.

167. The solid oral dosage form of claim 166, wherein the dosage form is a capsule or tablet.

168. A combination solid oral dosage form comprising: an opioid antagonist present in an amount from about 0.0001 mg to less than about 0.5 mg; an opioid agonist present in an amount from about 1.0 mg to about 300 mg; and at least one pharmaceutical excipient; wherein the solid dosage form comprises a suspension-filled soft gelatin capsule comprising: a soft gelatin capsule; and a suspension within the capsule comprising a non-aqueous vehicle in liquid form, the opioid agonist in solid form, and the opioid antagonist in solid form.

169. The solid oral dosage form of claim 168, wherein the suspension further comprises at least one pharmaceutical excipient.

170. A method for administering at therapeutic dose of an opioid antagonist to a human subject consisting essentially of administering a solid oral dosage form comprising an opioid antagonist present in an amount from about 0.0001 mg to less than about 0.5 mg, and an opioid agonist present in an amount from about 0.1 mg to about 300 mg.

171. A pharmaceutical kit comprising a dosage form comprising an opioid antagonist present in an amount from about 0.0001 mg to less than about 0.5 mg, and an opioid agonist present in an amount from about 0.1 mg to about 300 mg.
